(19) United States
(12) Patent Application Publication
(10) Pub. No.: US 2010/0105773 A1
(43) Pub. Date: Apr. 29, 2010

(54) USE OF RESOLVINS AND DOCOSATRIENES AND ANALOGUES THEREOF FOR THE TREATMENT OF ANGIOGENESIS AND OCULAR NEOVASCULARIZATION

(75) Inventors: Lois Smith, West Newton, MA (US); Kip Connor, Brighton, MA (US); Charles N. Serhan, Needham, MA (US)

Correspondence Address: DAVID S. RESNICK, NIXON PEABODY LLP, 100 SUMMER STREET, BOSTON, MA, 02110-2131, US

(73) Assignees: THE CHILDREN'S MEDICAL CENTER CORPORATION, Boston, MA (US); THE BRIGHAM AND WOMEN'S HOSPITAL, INC., Boston, MA (US)

(21) Appl. No.: 12/514,156

(22) PCT Filed: Nov. 9, 2007
(86) PCT No.: PCT/US2007/084306

§ 371 Date: Dec. 10, 2009

Related U.S. Application Data
(60) Provisional application No. 60/858,124, filed on Nov. 9, 2006.

(51) Int. Cl. A61K 31/202; A61P 19/02; A61P 17/02; A61P 17/06

(52) U.S. Cl. 514/560

(57) ABSTRACT

The present invention relates to methods and compositions for the treatment of, or prevention of angiogenesis in a subject. In particular, the present invention relates to methods to treat a subject with, or at risk of developing angiogenesis by administering a pharmaceutical composition comprising a resolvin or resolvin analogue or precursor, and/or a protectin or protectin analogue. In another embodiment, the present invention relates to the use of resolvins and protectins to treat pathologies associated with angiogenesis.

### CROSS REFERENCED APPLICATIONS

This application claims the benefit under 35 U.S.C. 119(e) of U.S. Provisional Application Ser. No. 60/858,124 filed on Nov. 9, 2006, the contents of which are incorporated herein by reference.

### GOVERNMENT SUPPORT

This invention was made with Government support under Grant Nos. EY008670, EY017017, EY14811, 5T32 EY07145, P50-DE016191, GM38765 awarded by the National Institutes for Health (NIH). The Government has certain rights in the invention.

### FIELD

The present invention relates to the use of di- and trihydroxy derivatives of EPA or DHA (resolvins or resolution phase interaction products) and/or DHA derivative 10,17S-docosatriene (Neuroprotectin D1) and their therapeutically stable analogues for the treatment of angiogenesis. The present invention also provides methods of preparation and package pharmaceuticals for use as medicaments for neovascularization and angiogenesis.

### BACKGROUND

Angiogenesis is a process of tissue vascularization that involves the growth of new developing blood vessels into a tissue, and is also referred to as neo-vascularization. Blood vessels are the means by which oxygen and nutrients are supplied to living tissues and waste products are removed from living tissue. When appropriate, angiogenesis is a critical biological process. It is essential in reproduction, development and wound repair. Conversely, inappropriate angiogenesis, also referred to as pathological angiogenesis, can have severe negative consequences. For example, it is only after many solid tumors are vascularized as a result of angiogenesis that the tumors have a sufficient supply of oxygen and nutrients that permit it to grow rapidly and metastasize.

As maintaining the rate of angiogenesis in its proper equilibrium is so critical to a range of functions, it must be carefully regulated in order to maintain health. Abnormal angiogenesis or pathological angiogenesis occurs when the body loses at least some control of angiogenesis, resulting in either excessive or insufficient blood vessel growth. Disease or disorders associated with pathological angiogenesis are those diseases and disorders which require or induce vascular growth. Such diseases represent a significant portion of all diseases for which medical treatment is sought, and include cancers, diabetic retinopathy, macular degeneration and inflammatory arthritis.

For instance, conditions such as ulcers, strokes, and heart attacks may result from the absence of angiogenesis normally required for natural healing. In contrast, excessive blood vessel proliferation can result in tumor growth, tumor spread, blindness, psoriasis and rheumatoid arthritis. Inhibition of angiogenesis is desirable. For example, in arthritis, new capillary blood vessels invade the joint and destroy cartilage. In diabetes, new capillaries invade the vitreous, bleed, and cause blindness. Ocular neovascularization is the most common cause of blindness. Tumor growth and metastasis are angiogenesis dependent. A tumor must continuously stimulate the growth of new capillary blood vessels for the tumor itself to grow.

Vessel loss and subsequent destructive neovascularization are also the two critical phases of many sight-threatening diseases. These include retinopathy of prematurity and diabetic retinopathy, leading causes of blindness in childhood and middle age and affecting over 4 million patients in the US. Ocular neovascularization (vessel loss) is the most common cause of blindness in all age groups, including diabetic retinopathy in working age-adults and age-related macular degeneration in the elderly, and are all conditions related to pathological angiogenesis in the eye.

Retinopathy of prematurity (ROP) is a potentially blinding disease, initiated by lack of retinal vascular growth after premature birth. The greatest risk factor for development of ROP is low birth weight and gestational age. Retinopathy of prematurity (ROP) is a potentially blinding eye disorder that primarily affects premature and underweight infants. The smaller a baby is at birth, the more likely that baby is to develop ROP. This disorder usually develops in both eyes, and is one of the most common causes of visual loss in childhood and can lead to lifelong vision impairment and blindness. About 1,100-1,500 infants annually develop ROP that is severe enough to require medical treatment. About 400-600 infants each year in the U.S. becomes legally blind from ROP. ROP occurs in two phases. (Simons, B. D. & Flynn, J. T. (1999) International Opthalmology Clinics 39, 29-48). When infants are born prematurely the retina is incompletely vascularized. In infants who develop ROP, growth of vessels slows or ceases at birth leaving maturing but avascular and therefore hypoxic peripheral retina. (Ashton, N. (1966) Am J Opthalmol 62, 412-35; Flynn, J. T., O′Grady, G. E., Herrera, J., Kushner, B. J., Cantolino, S. & Milam, W. (1977) Arch Opthalmol 95, 217-23). This is the first phase of ROP. The extent of non-perfusion of the retina in the initial phase of ROP appears to determine the subsequent degree of neovascularization, the late destructive stage of ROP, with the attendant risk of retinal detachment and blindness. (Penn, J. S., Tolman, B. L. & Henry, M. M. (1994) Invest Opthalmol V is Sci 35, 3429-35). If it were possible to allow blood vessels to grow normally in all premature infants, as they do in utero, the second damaging neovascular phase of ROP would not occur. When ROP was first described in 1942, the etiology was unknown. However, the liberal use of high supplemental oxygen in premature infants was soon associated with the disease and hyperoxia was shown to induce ROP-like retinopathy in neonatal animals with incompletely vascularized retinas. This suggested that an oxygen-regulated factor was involved. Expression of vascular endothelial growth factor (VEGF), which is necessary for normal vascular development, is oxygen-regulated and was found to be important for both phases of ROP. (Okamoto, N., Hofmann, F., Wood, J. M. & Campochiaro, P. A. (2000) American Journal of Pathology 156, 697-707). High supplemental oxygen affects the first phase of vascular growth in ROP animal models through suppression of VEGF expression. However, with current careful use of moderate oxygen supplementation, the oxygen level in patients is not a significant risk factor for ROP, yet the disease persists, suggesting that other factors are also involved. (Kinsey, V. E., Arnold, H. J., Kalina, R. E., Stem, L., Stahiman, M., Odell, G., Driscoll, J. M., Jr., Elliott, J. H., Payne, J. & Patz, A. (1977) Pediatrics 60, 655-68; Lucey, J. F. & Dangman, B. (1984) Pediatrics 73, 82-96).

Diabetic retinopathy is the most common diabetic eye disease and a leading cause of blindness in American adults. It is caused by changes in the blood vessels of the retina. In some cases of diabetic retinopathy, fragile, abnormal blood vessels develop and leak blood into the center of the eye, blurring vision. In others, abnormal new blood vessels grow on the surface of the retina.

Age-related macular degeneration is a degenerative condition of the macula (the central retina). It is the most common cause of vision loss in the United States in those 50 or older, and its prevalence increases with age of an individual. Age-related macular degeneration is caused by hardening of the arteries that nourish the retina. This deprives the sensitive retinal tissue of oxygen and nutrients that it needs to function and thrive. As a result, the central vision deteriorates. Ten percent of age related macular degeneration is caused by neovascularization, where new blood vessels form to improve the blood supply to oxygen-deprived retinal tissue.

The current treatment of diseases associated with abnormal or pathological angiogenesis are inadequate. Agents which prevent continued angiogenesis, e.g, drugs (TNP-470), monoclonal antibodies, antisense nucleic acids and proteins (angiostatin, endostatin and antiangiogenic ATIII) are currently being tested. Although preliminary results with the antiangiogenic proteins are promising, no one candidate has proven to possess all the qualities needed of an angiogenesis inhibitor. Thus, new agents that inhibit angiogenesis are needed, particularly for the treatment of ocular neovascularization, including for the treatment of retinopathy of prematurity. Control of angiogenesis in the eye can be approached by inhibition of neovascularization but control of vaso-obliteration which precedes this phase would also be desirable.

The role of protein growth factors in the regulation of angiogenesis is well known, but the role of lipids in this process, while beginning to be elucidated^{2,3}, is still largely undefined. Docosahexaenoic acid (DHA; C22:6omega-3) and arachidonic acid (AA; C20:4omega-6) are the major polyunsaturated fatty acids found in the retina⁴. DHA and AA are mainly found in neural and vascular cell membrane phospholipids and eicosapentaenoic acid (EPA; C20:5omega-3), the precursor to DHA, is found in retinal vascular endothelium⁵. Polyunsaturated fatty acids are released as free fatty acids by phospholipase A2, which is induced by ischemia, inflammation, neuroactive compounds, redox balance, and light exposure. Dietary sources of EPA, DHA, and AA contribute substantially with lipids from tissue to a substrate pool for enzymes that convert free polyunsaturated fatty acids to vaso- and immuno-regulatory lipid mediators⁶ which include separate families of bioactive mediators such as eicosanoids from AA, neuroprotectins such as neuroprotectin D1 from DHA, D series resolvins from DHA, and E series resolvins from EPA^{7,8}. Retinopathy and pathological angiogenesis can be modeled in the mouse eye with oxygen-induced vessel loss which precipitates hypoxia-induced retinopathy. This allows assessment of retinal vessel loss, vessel re-growth after injury and pathological angiogenesis¹. The use of lipids, in particular resolvins and protectins to treat vascular loss, vascular regrowth and angiogenesis has not been previously described.

### SUMMARY

The present invention relates to the use of di- and trihydroxy derivatives of EPA or DHA (resolvins or resolution phase interaction products) and/or the DHA derivative 10,17S-docosatriene (Neuroprotectin D1, also known as protectins) and their therapeutically stable analogues for the treatment and control of pathological angiogenesis. The present invention therefore, provides many new useful therapeutic applications for use of resolvins and neuroprotectins. In particular, the invention relates to the use of resolvins and protectins to treat pathologies associated with angiogenesis. In some embodiments, resolvins and protectins can be used to treat and/or reduce the risk of developing angiogenesis in the eye, for example but not limited to ocular neovascularization, for example retinopathy of prematurity (ROP), diabetic retinopathy and age related macular degeneration (AMD), and also angiogenesis associated with tumor growth.

The present invention relates to the discovery of the use of resolvins and protectins and their analogues as potent regulators of angiogenesis. In some embodiments, the angiogenesis is associated with ocular neovascularization. In some embodiments, the pathological angiogenesis is retinopathy of prematurity (ROP), age-related macular degeneration (AMD) and diabetic retinopathy. In alternative embodiments, the angiogenesis is associated with tumor growth. In alternative embodiments, the pathological angiogenesis is associated with arthritis and psoriasis.

One embodiment of the present invention is directed to pharmaceutical compositions of the resolvins and/or protectins of the invention for the treatment of angiogenesis. A further embodiment also provides methods to treat various diseases and disorders associated with pathological angiogenesis, for example ocular neovascularization. Examples of disease and disorders associated with ocular neovascularization are for example, but not limited to diabetic retinopathy, retinopathy of prematurity (ROP), age-related macular degeneration (AMD), retinal vein occlusion, radiation retinopathy. Angiogenesis is also associated with the pathogenesis of numerous diseases, including but not limited to tumor growth/metastasis, diabetic retinopathy, and in tissue remodeling upon injury and are also encompassed in this invention.

Another embodiment of the invention provides various methods to prepare pharmaceutical compositions comprising the resolvins and protectins of the invention. The invention also provides packaged pharmaceuticals comprising resolvins and protectins for use in the treatment and/or prevention of various diseases and disorders associated with angiogenesis.

While multiple embodiments are disclosed, still other embodiments of the invention will become apparent to those skilled in the art from following the detailed description. As will be apparent, the invention is capable of modifications in various aspects, all without departing from the scope of the spirit and scope of the present invention. Accordingly, the drawings and detailed description are to be regarded as illustrative in nature and nor restrictive.

One aspect of the present invention relates to a method for the treatment of, or reducing the risk of, developing angiogenesis in a subject, the method comprising administering to the subject an effective amount of a pharmaceutical composition comprising a resolvin or agonists or analogues or precursors thereof.

Another aspect of the present invention relates to a method for the treatment of, or reducing the risk of, developing angiogenesis in a subject, the method comprising administering to the subject an effective amount of a pharmaceutical composition comprising a protectin or agonists or analogues or precursors thereof.

In some embodiments, the resolvin is a di- or tri-hydroxy derivative of eicosapentaenoic acid (EPA) or deciashexaenoic acid (DHA). In some embodiments, the hydroxy derivative of eicosapentaenoic acid (EPA) or deciashexaenoic acid (DHA) is an E-series resolvin or 18R resolvin of the E series, for example but not limited to resolvin E1 (RvE1; (5S,12R,18R-trihydroxy-6Z,8E,10E,14Z,16E-eicosapentaenoic acid); 19-(p-fluorophenoxy)-RvE1; 18-oxo-RvE1; 5S,6R-epoxy, 18R-hydroxy-EPE, Resolvin E2 (RvE2).

In some embodiments, a hydroxy derivative of eicosapentaenoic acid (EPA) or deciashexaenoic acid (DHA) is a D series resolvin or a 17R or 17S resolvin of the D-series, for example but not limited to, 17R-diH DHA; 16,17R-diH DHA; 17R-H(p) DHA; 7(8)-epoxy-17R-DHA; 4(5)-epoxy-17R-H DHA; Resolvin D1 (17S,8,17R-triDHA); resolvin D2 (175,16,17-triDHA); Resolvin D3 (4S,11,17R-triDHA); Resolvin D4 (4S,5,17-triDHA).

In some embodiments, a protectin useful in the methods as disclosed herein is a di- or tri-hydroxy derivative of deciashexaenoic acid (DHA), for example but not limited to neuroprotectin D1 (NPD1); protectin D1 (PD1); 10,17s-docosatriene or analogues and mimetics of NPD1; PD1 or 10,17s-docosatriene.

In some embodiments, the resolvins and neuroprotectins useful in the methods and compositions as disclosed herein, and methods of their synthesis are disclosed in International Patent Applications WO04/014835 and WO05/105025 and U.S. Patent Applications 2005/0238589, 2006/0293288, 2005/0238589, 2005/0261255 and 2004/0116408 which are incorporated herein in their entirety by reference.

In some embodiments, a angiogenesis is associated with ocular neovascularization, tumor angiogenesis, arthritis, retinopathy, psoriasis, restenosis, capillary proliferation in atherosclerotic plaques. In some embodiments, retinopathy is, for example, but not limited to, retinopathy of prematurity (ROP); diabetic retinopathy; age-related macular degeneration (AMD); retina vein occlusion; sickle cell retinopathy; Stargardt's disease; choroidal neovascularization, radiation retinopathy; symptoms associated with microangiopathy, ocular neovascularization, neovascular glaucoma. In some embodiments, retinopathy is limited to; retinopathy of prematurity (ROP); diabetic retinopathy; retina vein occlusion; sickle cell retinopathy; choroidal neovascularization, radiation retinopathy; symptoms associated with microangiopathy, ocular neovascularization, neovascular glaucoma. In some embodiments, retinopathy is retinopathy of prematurity.

In some embodiments, the subject is born preterm, for example where a subject is born before full gestation or weighing 10% less that the average for the subjects gestation age.

Another aspect of the present invention relates to the use of a compound, for the manufacture of a medicament, for reducing the risk of retinal injury, when the retinal injury involves retinal occlusion followed by neovascularization, for example the use of a protectin and/or a resolvin. In such embodiments, a resolvin can be a di- or tri-hydroxy derivative of eicosapentaenoic acid (EPA) or deciashexaenoic acid (DHA). In some embodiments, a hydroxy derivative of eicosapentaenoic acid (EPA) or deciashexaenoic acid (DHA) is an E-series resolvin or 18R resolvin of the E series, for example, but not limited to, an E-series resolvin is selected from a group comprising; resolvin E1 (RvE1; (5S,12R,18R)-trihydroxy-6Z,8E,10E,14Z,16E-eicosapentaenoic acid); 19-(p-fluorophenoxy)-RvE1; 18-oxo-RvE1; 5S,6R-epoxy, 18R-hydroxy-EPE, Resolvin E2 (RvE2). In some embodiments, the present invention use of a hydroxy derivative of eicosapentaenoic acid (EPA) or deciashexaenoic acid (DHA), such as a D series resolvin or a 17R or 17S resolvin of the D-series, for example, but not limited to, D resolvins 17R-diH DHA; 16,17R-diH DHA; 17R-H(p) DHA; 7(8)-epoxy-17R-DHA; 4(5)-epoxy-17R-H DHA; Resolvin D1 (17S,8,17R-triDHA); resolvin D2 (17S,16,17-triDHA); Resolvin D3 (4S,11,17R-triDHA); Resolvin D4 (4S,5,17-triDHA). In some embodiments, the protectin is a di- or tri-hydroxy derivative of deciashexaenoic acid (DHA). In some embodiments, the hydroxy derivative of DHA is selected from a group comprising; neuroprotectin D1 (NPD1); protectin D1 (PD1); 10,17s-docosatriene or analogues and mimetics of NPD1; PD1 or 10,17s-docosatriene. In some embodiments, the resolvins and neuroprotectins useful in the methods and compositions as disclosed herein, and methods of their synthesis are disclosed in International Patent Applications WO04/014835 and WO05/105025 and U.S. Patent Applications 2005/0238589, 2006/0293288, 2005/0238589, 2005/0261255 and 2004/0116408 which are incorporated herein in their entirety by reference.

Another aspect of the present invention relates to an article of manufacture comprising packaging material and a pharmaceutical agent contained within the packaging material, wherein the packaging material comprises a label which indicates the pharmaceutical may be administered, for a sufficient term at an effective dose, for treating or reducing the risk of angiogenesis, wherein the pharmaceutical agents comprises a resolvin and/or protectin with a pharmaceutically effective carrier. In some embodiments, the angiogenesis is associated with ocular neovascularization, tumor angiogenesis, arthritis, retinopathy. In some embodiments, the resolvin is Resolvin E (RvE1) and/or Resolvin D1 (RvD1), or a protectin, as disclosed herein, for example but not limited to, neuroprotectin D1 (NPD1).

In some embodiments, administration comprises intravenous, transdermal, instrasynovial, intramuscular, or oral administration. In some embodiments, a resolvin or agonist or analogue or precursor thereof are administered prophylatically, an in some embodiments, administration is therapeutically and/or prophylatically. In some embodiments, administration is conducted in conjunction with known angiogenesis inhibitors and/or other therapeutic treatments.

In some embodiments, subjects amenable to administration are subjects at risk of developing a disease or disorder associated with angiogenesis, for example, subject identified to be at risk of developing a disease or disorder associated with angiogenesis is determined genetically. In some embodiments, a subject is identified to be at risk of developing a disease or disorder associated with angiogenesis by measuring for a biomarker that identifies subjects afflicted with, or at risk of developing a disease or disorder associated with angiogenesis.

### BRIEF DESCRIPTION OF THE DRAWINGS

FIG. 1 shows a collection of photomicrographs (a and d) bar graphs (b, c, e-h) and schematics (i). Retinas from C57B1/6 mice fed a diet with a physiologic elevation in either omega-3 or omega-6 polyunsaturated fatty acids were isolated, stained, and flat-mounted after induction of retinopathy. Panel 1a shows P17 retinal vasculature stained with lectin-FITC showed more extensive vaso-obliteration (†) and neovascularization (††) in the omega-6 versus omega-3 polyunsaturated fatty acid fed mice (omega-6 n=14, and omega-3 n=27). Panel 1b shows vaso-obliteration (†p≦0.0001) and panel 1c shows neovascularization (††p≦0.0001) was reduced ±2 fold in the omega-3 versus omega-6 polyunsaturated fatty acid fed mice at P17. After induction of retinopathy, retinas from fat-1 homozygotes and wild type control mice were flat-mounted at P17. Panel 1d shows retinal vasculature stained with lectin-FITC shows more extensive retinal vaso-obliteration (*) and neovascularization (**) in wild type versus fat-1 retinas. Panel 1e shows vaso-obliteration was reduced ±2 fold (*p≦0.001) and panel 1f shows neovascularization reduced ±2 fold (**p≦0.001) in the fat-1 expressing mice with higher levels of omega-3 polyunsaturated fatty acids and lower levels of omega-6 polyunsaturated fatty acids compared to controls (WT n=20, and Fat-1 n=16). Panel 1g and 1 h shows that after exposure to 75% oxygen for 24 hours from P7-P8, retinas from either C57B1/6 mice given a diet high in either omega-3 or omega-6 polyunsaturated fatty acids (omega-6 n=10, and omega-3 n=10) (1g) or Fat-1 mice and controls (WT n=14, and Fat-1 n=7) (1h) shows equally extensive vaso-obliteration in both the (1g) omega-6 polyunsaturated fatty acid and omega-3 polyunsaturated fatty acid fed mice as well as in the (1h) Fat-1 and wild type controls at P8. Panel 1i shows resolvins and Neuroprotectins Biosynthetic Schematic from omega-3 polyunsaturated fatty acid. The omega-22 hydroxy-PD1 is the inactivation metabolic of NPD1, a biosynthetic marker of this pathway. RvE2 of the E series EPA resolvins is also a biosynthetic marker identified in the retina.

FIG. 2 shows a collection of data presented in various forms. Panel 2a shows two spectra, a LC MS/MS spectrum of RvE₂ and a spectrum of omega-22-hydroxy-PD1 obtained from retinal extracts of mice given an omega-3 polyunsaturated fatty acid diet. Panel 2b shows a tabulation of relative levels of RvE₂ and omega-22-hydroxy-PD1 in retinas of mice on a high omega-3 polyunsaturated fatty acid diet (6 retinas). Neither resolvins nor neuroprotectins were identified in retinas of omega-6 polyunsaturated fatty acid fed mice. C57B1/6 mice were injected i.p. daily P6-P17 with 10 ng of RvD1, RvE1, NPD1 or a Saline/EtOH control (RvD1 n=14, RvE1 n=10, NPD1 n=14 and Saline n=14). Panel 2c shows a bar graph which compares vessel loss in RvD1, RvE1 or NPD1 treated mice compared to their vehicle control treated counterparts. A 40% decrease in vessel loss (VO) was observed in RvD1, RvE1 or NPD1 treated mice compared to their vehicle control treated counterparts (*p≦0.001). Panel 2d shows a bar graphs which compares neovascularization observed in mice injected i.p. with RvD1, RvE1, or NPD1 compared to vehicle-treated mice (†p≦0.03). There was a 30% decrease in neovascularization (Tufts) in mice injected i.p. with RvD1, RvE1, or NPD1 compared to vehicle-treated mice (†p≦0.03). Panel 2e shows a bar graph which compares vaso-obliteration (VO) in mice injected from P5-P8 as above. There was no protective action with either RvE1 or NPD1 treatment on oxygen-induced vessel loss at P8 (RvD1 n=7, RvE1 n=9, NPD1 n=7 and Saline n=6).

FIG. 3 shows a collection of bar graphs (a-f), one (3b) also having a photo of a Western blot probed for TNF-α. Panel 3a shows that mean total retinal TNF-α mRNA expression was increased at P8 and P14 approximately 10-fold in omega-6 fed mice compared to their omega-3 polyunsaturated fatty acid fed counterparts (*p≦0.0001, n=4). Panel 3b shows retinal levels of TNF-α were analyzed by Western blot analysis in mice on either omega-3 polyunsaturated fatty acid or omega-6 polyunsaturated fatty acid diets. Mice on the omega-3 polyunsaturated fatty acid diet had a significant decrease in TNF-α protein levels (#p≦0.001, n=4). Panel 3c shows intraperitoneal injections of a TNF-α receptor fusion protein (etanercept) resulted in a significant reduction in vaso-obliteration in omega-6 polyunsaturated fatty acid fed mice compared to saline injected controls (†p≦0.001, n=8). Panel 3d shows TNF-α receptor fusion protein treated omega-6 polyunsaturated fatty acid fed mice had a significant reduction in pathologic neovascularization compared to saline injected controls (††p≦0.05, n=8). Panel 3e shows intraocular injections of the TNF-α receptor fusion protein significantly reduce vaso-obliteration compared to fellow saline-injected eye in omega-6 polyunsaturated fatty acid fed mice (p≦0.005, Saline n=10 and anti-TNF-α n=7). Panel 3f shows intraocular administration of the TNF-α receptor fusion protein also significantly improved neovascularization in these mice (p≦0.05, Saline n=10 and anti-TNF-α n=7).

### DETAILED DESCRIPTION

The present invention relates to methods and methods for the prevention and treatment of angiogenesis. In particular, the present invention is based, in part on the discovery that in a model of angiogenesis, in particular a model of hypoxia-induced retinopathy, resolvins and protectins can prevent vessel loss (vasoobliteration) and vessel regrowth (neovascularization).

The inventors have discovered that the resolvins, in particular Resolvin D1 (RvD1) and resolvin E1 (RvE1) and a DHA metabolite, 10, 17S— docosatriene (“Neuroprotectin D1” or “NPD1”), provides surprisingly effective prevention of neovascularization and pathological angiogenesis when administered after an ischemia-induced retinopathy. Moreover, both resolvins RvE1 and RvD1 and 10,17S-docosatriene (NPD1) potently counteracted TNF oxidative stress-mediated cell apoptotic damage. Overall, RvE1, RvD1 and NPD1 protected against neovascularization in a model of hypoxia induced retinopathy, and will be important in protecting against neovascularization in a number of disease and disorders such are retinopathy of prematurity (ROP) and diabetic retinopathy. In one embodiment of the invention, subjects at risk of developing angiogenesis and/or pathological angiogenesis are treated with the resolvins and/or protectins and their analogues or precursors of the present invention.

Resolvins are natural counter regulatory lipid mediators in host defense mechanisms that protect the host tissues from effector cell mediated injury, for example they function as counteregulatory mediators. For example, the protect cells from over amplification of specific protective mechanisms. For example, while wishing not to be bound by theory, it is believed that resolvins protect cells from excessive vessel regrowth (neovascularization) or pathological angiogenesis which are protective mechanisms following vessel loss (vasoobliteration), which can occur due to numerous insults, for example but not limited to oxygen-induced vessel loss. Some diseases characterized by angiogenesis may represent the loss of and/or genetically determined low resolvin endogenous responders and/or levels. The resolvin and protectin analogues described throughout the application can be used to replace, enhance and/or treat the loss of these resolvins and protectins therapeutically and thereby pharmaceutically resolve pathological angiogenesis. Resolvins useful in the present invention are discussed in U.S. Patent Application 2004/0116408, 2005/0261255 and PCT application 06/06075457 and are incorporated herein in their entirety by reference.

10,17S-docosatriene (also referred to as neuroprotectin D1 (NPD1) herein) is a dihydroxy-containing DHA derivative and is also useful in the present invention and is discussed in Hong, S., Gronert, K., Devchand, P. R., Moussignac, R. L. and Serhan, C. N. (2003) J. Biol. Chem. 278:14677-14687; and International Application No. WO 04/014835, the contents of which are incorporated herein by reference in their entirety. Although the precise enzymes involved in NPD1 synthesis have not been identified, mounting evidence suggests that a PLA2 enzymatic reaction which is then followed by a 15-lipoxygenase-like reaction is involved. PLA2, which liberates free DHA from membrane phospholipids, and a 15-LOX-like activity, which converts DHA into NPD1 are prime candidates; and Serhan, C. N., Clish, C. B., Brannon, J., Colgan, S., Chiang, N., and Gronert, K. (2000) J. Exp. Med., 192, 1197-1204; Serhan, C. N., Hong, S., Gronert, K., Colgan, S. P., Devchand, P. R., Mirick, G., and Moussignac, R. L. (2002) J; Exp. Med. 196, 1025-1037; and Hong, S., Gronert, K., Devchand, P. R., Moussignac, R. L., and Serhan, C. N. (2003) J. Biol. Chem. 278, 14677-14687.

In some embodiments, the resolvins and neuroprotectins useful in the methods and compositions as disclosed herein, and methods of their synthesis are disclosed in International Patent Applications WO04/014835 and WO05/105025 and U.S. Patent Applications 2005/0238589, 2006/0293288, 2005/0238589, 2005/0261255 and 2004/0116408 which are incorporated herein in their entirety by reference.

Definitions. For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “EPA” or “eicosapentaenoic acid” are used interchangeably herein. The term “DHA” or “docosahexaenoic acid” are used interchangeably herein, refer to 4,7,10,13,16,19-docosahexaenoic acid. The term “PUFA” or “polyunsaturated fatty acids” are used interchangeably herein.

“Preterm” or “preterm birth” or “prematurity” refers to birth of a patient prior to 40 weeks of gestation or weighing 10% less than the average for the patient's gestational age.

As used herein, the term “subject” refers to any living organism in which angiogenesis and/or pathological angiogenesis can be elicited. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection.

The terms “subject” and “individual” are used interchangeably herein, and refer to an animal, for example a human, to whom treatment, including prophylactic treatment, with the pharmaceutical composition according to the present invention, is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term ‘effective amount” as used herein refers to the amount of therapeutic agent of pharmaceutical composition to alleviate at least some of the symptoms of the disease or disorder.

As used herein, the term “treating” includes reducing or alleviating at least one adverse effect or symptom of a angiogenesis or condition, disease or disorder associated with angiogenesis and/or pathological angiogenesis, i.e., disorders characterized by abnormal angiogenesis.

As used herein, the terms “administering,” and “introducing” are used interchangeably herein and refer to the placement of the resolvins and protectins of the invention into a subject by a method or route which results in at least partial localization of resolvins and protectins at a desired site. The resolvins and or protectins can be administered by any appropriate route which results in an effective treatment in the subject.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The articles “a” and “an” are used herein to refer to one or to more than one (i.e., to at least one) of the grammatical object of the article. By way of example, “an element” means one element or more than one element.

Therapeutic Compositions

Resolvins

Resolvins are natural counter regulatory lipid mediators in host defense mechanisms that protect the host tissues from effector cell mediated injury, for example they function as counteregulatory mediators. For example, the protect cells from over amplification of specific protective mechanisms. The inventors of the present invention have discovered that resolvins protect cells from excessive vessel regrowth (neovascularization) or angiogenesis which in some instances is a protective mechanisms following vessel loss (vasoobliteration), which can occur due to numerous insults, for example but not limited to oxygen-induced vessel loss. Some diseases characterized by angiogenesis may represent the loss of and/or genetically determined low resolvin endogenous responders and/or levels. The resolvin and protectin analogues described throughout the application can be used to replace, enhance and/or treat the loss of these resolvins and protectins therapeutically and thereby pharmaceutically resolve angiogenesis. The resolvins that are useful in the present invention are discussed in U.S. Patent Application 2004/0116408, 2005/0261255 and PCT application 06/06075457 and are incorporated herein in their entirety by reference.

In some embodiments, the resolvins and neuroprotectins useful in the methods and compositions as disclosed herein, and methods of their synthesis are disclosed in International Patent Applications WO04/014835 and WO05/105025 and U.S. Patent Applications 2005/0238589, 2006/0293288, 2005/0238589, 2005/0261255 and 2004/0116408 which are incorporated herein in their entirety by reference.

Resolvins derived from EPA are designated the E series, while resolvins derived from the precursor DHA are designated the D series, also referred to as Resolvin D1-D4, or RvD1 to RvD4.

Resolvins, such as resolvin E1 (RvE1; 5S,12R,18R-trihydroxyeicosapentaenoic acid) are novel anti-angiogenic lipid mediators derived from omega-3 fatty acid eicosapentaenoic acid (EPA). At the site of angiogenesis, EPA can be converted to 18R-oxygenated products including RvE1 that carry potent anti-angiogenic signals.

Surprisingly, resolvins (the compounds identified throughout the application) such as RvE1 and RvD1 protected against the development hypoxia induced retinal vasoobliteration and neovascularization in a mouse model of retinopathy of prematurity and angiogenesis.

Resolvin E1 (RvE1) and Resolvin D1 (RvD1) are endogenous lipid mediators that belong to an array of natural bioactive lipids that are generated in vivo from c-3 polyunsaturated fatty acids (PUFA), such as omega-3 (or co-3), eicosapenaenic acid (EPA) and docosahexaenoic acid (DHA) by aspirin modified COX-2 (Serhan, C. N., et al. (2000) J. Exp. Med. 192, 1197; Serhan, C. N., et al. (2002) J. Exp. Med. 196, 1025, the contents of which are incorporated herein by reference). The use of resolvin E1 (RvE1) and resolvin D1 (RvD1) is further described in Example 2. In certain embodiments, the compounds of the invention are prepared in vivo or in vitro and then substantially purified and isolated by techniques known by persons in the art (see, for example, U.S. Pat. No. 6,670,396, the contents of which are incorporated herein by reference). Without limitation, the purity of the compounds is generally at least about 90%, preferably at least about 95%, and most preferably at least about 99%. Certain compounds of the invention may also be prepared by chemically modifying one or more purified compounds. For example, a purified compound may be chemically modified into a pharmaceutically acceptable salt or prodrug as described above. Additionally or alternatively, one or more hydroxyl, thiol or amino groups may be protected as further described below. Additionally, in other embodiments, the compounds of formula I are manufactured independently using conventional synthetic methods for preparing lipid derivatives.

As mentioned above, the resolvins of this invention can be prepared by methods provided in U.S. patent application Ser. Nos. 09/785,86, filed Feb. 16, 2001, entitled “Aspirin Triggered Lipid Mediators” by Charles N. Serhan and Clary B. Clish, 10/639,714, filed Aug. 12, 2003, entitled “Resolvins: Biotemplates for Novel Therapeutic Interventions” by Charles N. Serhan and PCT Applications WO 01/60778, filed Feb. 16, 2001, entitled “Aspirin Triggered Lipid mediators” by Charles N. Serhan and Clary B. Clish and WO 04/014835, filed Aug. 12, 2003, entitled “Resolvins: Biotemplates for Novel Therapeutic Interventions” by Charles N. Serhan, the contents of which are incorporated herein by reference in their entirety.

In some embodiments, the resolvins used in the present invention for the treatment of angiogenesis are E-series resolvins, also known as 18R Resolvins of the E-series. In some embodiments the resolvin is Resolvin E1 (RvE1) also referred to as 5S,12R,18R-trihydroxy-6Z,8E,10E,14Z,16E-eicosapentaenoic acid or 5S,12R,18R-trihydroxy-EPE or, which is product of the conversion of EPA (see Gilroy et al, Nature Reviews, 2004; 3; 401-416). In other embodiments, the resolvin is Resolvin E2 (RvE2) also referred to as 15S,18R-dihydroydroxy-EPE. Also encompassed for the treatment of angiogenesis in the present invention are analogues and precursors and intermediates of resolvins of the E series, for example, but not limited to 19-(p-fluorophenoxy)-RvE1; 18-oxo-RvE1; and 5S,6R-epoxy,18R-hydroxy-EPE, 18R-hydro(per)oxy-EPE and 5S-hydroperoxy,18R-hydroxy-EPE.

By way of background, the biosynthesis of 18R resolvins of the E-series involves the cyclooxygenase enzyme COX2 that has been acetylated by aspirin, which introduces an 18R hydroperoxy-group into the EPA molecule. This is reduced to the corresponding hydroxy compound before a 5S-hydroperoxy group is introduced into the molecule, and a further reduction step produces 15S,18R-dihydroxy-EPE or resolvin E2 (RvE2). Alternatively, the 5S-hydroperoxy,18R-hydroxy-EPE intermediate is converted to a 5,6-epoxy fatty acid in polymorphonuclear neutrophils in humans and eventually to 5S,12R,18R-trihydroxy-6Z,8E,10E,14Z,16E-eicosapentaenoic acid or resolvin E1 (RvE1).

In alternative embodiments, the resolvins used in the present invention for the treatment of angiogenesis are D-series resolvins, also known as a 17R and 17S-resolvins of the D resolvin series (see Gilroy et al, Nature Reviews, 2004; 3; 401-416). In one embodiment, the D-series resolvin is Resolvin D1 (RvD1) also referred to as 17S,8,17R-triDHA, which is a product of the conversion of DHA to 17R-H(p)DHA to 7(8)-epoxy17R-DHA (see Gilroy et al, Nature Reviews, 2004; 3; 401-416). In another embodiment, the D-series resolvin is Resolvin D2 (RvD2) also referred to as 17S,16,17R-trihydroxyDHA, which is also a product of the conversion of DHA to 17R-H(p)DHA to 7(8)-epoxy17R-DHA.

Also encompassed for use in the present invention for the treatment of angiogenesis are analogues, intermediates and precursors of the D-series of resolvins, for example but not limited to other D-series resolvins such as Resolvin D3 (4S,11,17R-triDHA); Resolvin D4 (4S,5,17-triDHA) and their analogues and precursors for example but not limited to 10,17R-diH DHA; 16,17R-diH DHA; 17R-H(p) DHA; 7(8)-epoxy-17R-DHA; and 4(5)-epoxy-17R-H DHA.

By way of background, the biosynthesis of D-series of resolvins involves the conversion of DHA to 17R-D-series resolvins by a similar aspirin-triggered mechanism as EPA conversion to E-series resolvins. In the absence of aspirin, COX-2 converts DHA to 13S-hydroxy-DHA. However, in the presence of aspirin, DHA is converted to 17R-hydroxy-DHA, which is further converted to 7S-hydroperoxy,17R-hydroxy-DHA (17R-H(p)DHA) by a lipoxygenase enzyme and is further converted to an epoxy intermediate (7(8)-epoxy-17R-DHA) to be converted to Resolvins D1 (RvD1 or 7S,8,17R trihydroxyDHA) and Resolvin D2 (RvD2 or 17S,16,17R-trihydroxyDHA). Resolvins D1 and Resolvin D2 are 17S-Resolvins of the D series which are produced in cells in the absence of aspirin by a reaction catalyzed in the first step by a lipoxygenase. An alternative lipoxygenase-generated epoxy intermediate, 4S-hydroperoxy,17R-hydroxy-DHA, is transformed via an epoxide to Resolvins D3 (RvD3 or 4S,11,17R-trihydroxyDHA) and Resolvin D4 (RvD4 or 4S,5,17R-trihydroxyDHA).

Protectins

Protectin, or neuroprotectins (used interchangeably herein) are oxygenated metabolites of DHA that comprise conjugated triene structures, or docosatrienes (DT). One such dihydroxy-containing DHA derivative is termed neuroprotectin or Neuroprotectin D1 (NPD1) or 10,17S-docosatriene herein. The biosynthetic pathway of neuroprotectin or NPD1 also referred to herein as protectin involves the conversion of DHA in the presence of aspirin to a 17S-hydroperoxy-DHA, which is further converted to the 16(17)-epoxide (or 16,17-epoxy-docosatriene) intermediate and then to the 10,17S-dihydroxy docosatriene, denoted as 10,17S-DT or NPD1. See, Hong, S., Gronert, K., Devchand, P. R., Moussignac, R. L. & Serhan, C. N. (2003) J. Biol. Chem. 278:14677-14687; and International Application No. WO 04/014835, the contents of which are incorporated herein by reference in their entirety. Although the precise enzymes involved in NPD1 synthesis have not been identified, mounting evidence suggests that a PLA2 enzymatic reaction which is then followed by a 15-lipoxygenase-like reaction is involved. PLA2, which liberates free DHA from membrane phospholipids, and a 15-LOX-like activity, which converts DHA into NPD1 are prime candidates and Serhan, C. N., Clish, C. B., Brannon, J., Colgan, S., Chiang, N., and Gronert, K. (2000) J. Exp. Med., 192, 1197-1204; Serhan, C. N., Hong, S., Gronert, K., Colgan, S. P., Devchand, P. R., Mirick, G., and Moussignac, R. L. (2002) J; Exp. Med. 196, 1025-1037; and Hong, S., Gronert, K., Devchand, P. R., Moussignac, R. L., and Serhan, C. N. (2003) J. Biol. Chem. 278, 14677-14687.

In some embodiments, the protectins used in the present invention for the treatment of angiogenesis are neuroprotectin, neuroprotectin D1 (NPD1); protectin D1 (PD1); 10,17s-docosatriene or analogues and mimetics of NPD1; PD1 or 10,17s-docosatriene.

In some embodiments, the resolvins and neuroprotectins useful in the methods and compositions as disclosed herein, and methods of their synthesis are disclosed in International Patent Applications WO04/014835 and WO05/105025 and U.S. Patent Applications 2005/0238589, 2006/0293288, 2005/0238589, 2005/0261255 and 2004/0116408 which are incorporated herein in their entirety by reference.

Method of Treatment of a Subject

The present invention relates generally to a method of inhibiting angiogenesis in a mammal having an angiogenic disease or disorder. The method of the present invention comprises the administration of an effective amount of resolvin and/or protectin of the invention or analogue thereof having antiangiogenic activity to a mammal. This discovery is important because of the role that angiogenesis plays in a variety of disease processes. By inhibiting angiogenesis, one can intervene in the disease, ameliorate the symptoms, and in some cases cure the disease.

Where the growth of new blood vessels is the cause of, or contributes to, the pathology associated with a disease, inhibition of angiogenesis will reduce the deleterious effects of the disease. Examples include rheumatoid arthritis, retinopathies, diabetic retinopathy, inflammatory diseases, restenosis, and the like. Where the growth of new blood vessels is required to support growth of a deleterious tissue, inhibition of angiogenesis will reduce the blood supply to the tissue and thereby contribute to reduction in tissue mass based on blood supply requirements. Examples include growth of tumors where neovascularization is a continual requirement in order that the tumor grows beyond a few millimeters in thickness, and for the establishment of solid tumor metastases.

The invention provides for a method for the inhibition of angiogenesis in a tissue, and thereby inhibiting events in the tissue which depend upon angiogenesis. Generally, the method comprises administering to the subject and/or the tissue a composition comprising a therapeutic effective amount of a resolvin and/or protectin on the invention.

As described herein, pathological angiogenesis can occur in a variety of tissues, or organs comprised of organized tissues, including skin, muscle, gut, connective tissue, joints, bones and the like tissue in which blood vessels can invade upon angiogenic stimuli. As described earlier, angiogenesis includes a variety of processes involving neovascularization of a tissue including “sprouting”, vasculogenesis, or vessel enlargement. With the exception of traumatic wound healing, corpus leuteum formation and embryogenesis, it is believed that the majority of angiogenesis processes are associated with disease processes and therefore the use of the present therapeutic methods are selective for the disease and do not have deleterious side effects.

There are a variety of diseases or disorders in which angiogenesis is believed to be important, referred to as angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

Thus, methods which inhibit angiogenesis in a diseased tissue ameliorates symptoms of the disease and, depending upon the disease, can contribute to cure of the disease. In one embodiment, the invention contemplates inhibition of angiogenesis, per se, in a tissue. The extent of angiogenesis in a tissue, and therefore the extent of inhibition achieved by the present methods, can be evaluated by a variety of methods, for example such as are described in the Examples quantification of vaso-obliteration and retinal neovascularization.

In an important embodiment of the invention, a tissue to be treated is a retinal tissue of a subject with a retinal disease and/or ocular neovascularization, such as but not limited to diabetic retinopathy, age related macular degeneration (AMD), retinopathy of prematurity (ROP), or neovascular glaucoma and the angiogenesis to be inhibited is retinal tissue angiogenesis where there is neovascularization of retinal tissue.

In some embodiments, where the treatment is to reduce the risk of for the treatment of retinopathy of prematurity, the treatment administration of the resolvins and/or protection of the invention can be initiated soon after birth in order to effectively prevent complications of prematurity and to promote normal vascular development. This is especially critical for the treatment of ROP, wherein treatment which is delayed until after the non-vascularized retina becomes hypoxic might trigger abnormal retinal neovascularization.

In embodiments where the neovascularization is ocular neovascularization, for example diabetic retinopathy, age-related macular degeneration (AMD) and retinopathy of prematurity (ROP), the pharmaceutical composition of the invention may be administered to the subject prophylatically, for instance, if the subject has been identified to be at risk of developing retinopathies, and/or ocular neovascularization, for example diabetic retinopathies, AMD and ROP.

Thus, in one related embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In a related embodiment, a tissue to be treated is a tumor tissue of a subject with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the pharmaceutical composition of the invention, includes for example, but not limited to tumors of the lung, pancreas, breast, colon, laryngeal, ovarian, and the like tissues. In some embodiment, the solid tumor tissue treatable by the present methods include thyroid, and the cancer type is medullary thyroid cancer. Inhibition of tumor tissue pathological angiogenesis is important due to the role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

The methods are also effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the practice of the method in conjunction with other therapies such as conventional chemotherapy directed against solid tumors and for control of establishment of metastases. The administration of angiogenesis inhibitor is typically conducted during or after chemotherapy, although it is also encompassed within the present invention to inhibit angiogenesis after a regimen of chemotherapy at times where the tumor tissue will be responding to the toxic assault by inducing angiogenesis to recover by the provision of a blood supply and nutrients to the tumor tissue. In addition, the pharmaceutical compositions of the invention for the treatment of tumor-associated angiogenesis can be administrated prophylatically and/or before the development of a tumor, if the subject has been identified as to have a risk of developing cancer, for example to subjects that are positive for biomarkers of cancer cells or tumors. Insofar as the present methods apply to inhibition of tumor neovascularization, the methods can also apply to inhibition of tumor tissue growth, to inhibition of tumor metastases formation, and to regression of established tumors.

The inventive methods disclosed herein provide for the parenteral an oral administration of resolvins and/or protectins, or stable analog thereof in combination with other pharmaceutical compositions to infants in need of such treatment. Parenteral administration includes, but is not limited to, intravenous (IV), intramuscular (IM), subcutaneous (SC), intraperitoneal (IP), intranasal, and inhalant routes. In the method of the present invention, the resolvins and/or protectins or analogs thereof are preferably administered orally. IV, IM, SC, and IP administration may be by bolus or infusion, and may also be by slow release implantable device, including, but not limited to pumps, slow release formulations, and mechanical devices. The formulation, route and method of administration, and dosage will depend on the disorder to be treated and the medical history of the patient. In general, a dose that is administered by subcutaneous injection will be greater than the therapeutically-equivalent dose given intravenously or intramuscularly. Preferably, the dose of resolvin and/or protectin or analogs thereof administered will be from about 0.01 mg/kg to about 20 mg/kg of body weight. More preferably, the dose of resolvin and/or protectin or analogues thereof will be from about 1 mg/kg to about 10 mg/kg.

The present method for inhibiting angiogenesis in a tissue, and therefore for also practicing the methods for treatment of angiogenesis-related diseases, comprises contacting a tissue in which angiogenesis is occurring, or is at risk for occurring, with a composition comprising a therapeutically effective amount of resolvin and/or protectin and/or analogues thereof.

The subject treated in the present invention in its many embodiments is desirably a human subject, although it is to be understood that the principles of the invention indicate that the invention is effective with respect to all mammals. In this context, a mammal is understood to include any mammalian species in which treatment of diseases associated with angiogenesis is desirable, particularly agricultural and domestic mammalian species.

Administration of Pharmaceutical Compositions

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

After formulation with an appropriate pharmaceutically acceptable carrier in a desired dosage, the pharmaceutical compositions of this invention can be administered to a subject. The pharmaceutical compositions of this invention can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

When the resolvins and/or protectins of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one EPA or DHA analog, in combination with a pharmaceutically acceptable carrier.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 0.1 to about 40 mg per kg per day. For example, between about 0.01 microgram and 20 micrograms, between about micrograms and 100 micrograms and between about 10 micrograms and 200 micrograms of the compounds of the invention are administered per 20 grams of subject weight.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the resolvins and/or protectins or analogues thereof of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the resolvins and/or protectins or analogues thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

A prophylatically or therapeutically effective amount is also one in which any toxic or detrimental effects of the compound are outweighed by the beneficial effects.

Dosage regimens may be adjusted to provide the optimum desired response (e.g. a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

“Dosage unit” form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the EPA or DHA analog and the particular therapeutic or prophylactic effect to be achieved, and (b) the limitations inherent in the art of compounding such an active compound for the treatment of sensitivity in individuals.

The therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is sufficient to reduce inflammation and bone loss in a subject suffering froth a periodontal disease. In preferred embodiments, the therapeutically effective amount is sufficient to eliminate inflammation and bone loss in a subject suffering from a periodontal disease.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

An exemplary, non-limiting range for a therapeutically or prophylactically effective amount of the resolvins and/or protectins or analogues thereof of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Delivery of the resolvin and/or protectin and analogs thereof of the present invention to the eye is an important method of treating a variety of ocular neovascularization disease and disorders noted throughout the specification, including such common local conditions as diabetic retinopathy and age-related macular degeneration (AMD). The resolvins and/or protectins or analogues thereof can be administered to the eye in the form of an aerosol of particles, or eye ointments or solutions or any other means known by persons skilled in the art. In some embodiments, the delivery is via eye drops or ointments. In other embodiments aerosol formulation can be presented as a liquid or a dry powder, by method commonly known in the art.

The invention features an article of manufacture that contains packaging material and the resolvins and/or protectins or analogues thereof in a formulation contained within the packaging material. This formulation contains an at least one resolvins and/or protectins or analogues thereof and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference. Suitable the resolvins and/or protectins or analogues thereof are described herein.

More specifically, the invention features an article of manufacture that contains packaging material and at least one resolvins and/or protectins or analogues contained within the packaging material. The packaging material contains a label or package insert indicating that the formulation can be administered to the subject to alleviate angiogenesis, for example but not limited to ocular neovascularization, for example for the treatment of retinopathy of prematurity (ROP) o or diabetic retinopathy etc. in an amount, at a frequency, and for a duration effective treat or prevent symptoms associated with such disease states or conditions discussed throughout this specification.

Pharmaceutical Compositions

While it is possible for compounds of the present invention, for example the resolvins and/or protectins and analogues or precursors thereof, to be administered alone, it is preferable to administer the compound as a pharmaceutical composition.

Formulations of the invention can be prepared by a number or means known to persons skilled in the art. In some embodiments the formulations can be prepared by combining (i) at least one EPA or DHA analog in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, that comprise one or more resolvins and/or protectins or their analogues of the invention.

In certain embodiments, the resolvin and/or protectin are administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are antimicrobial compounds and/or non-steroidal antiinflammatory agents (NSAIDs). In certain preferred embodiments, the additional therapeutic agent or agents are COX-2 inhibitors, preferably selective COX-2 inhibitors, e.g. celecoxib, rofecoxib, and/or valdecoxib.

In some embodiments the pharmaceutical composition comprises resolvins and/or protectins and their precursors and/or analogues, alone or in any plurality of combinations. In other embodiments, the pharmaceutical compositions optionally further comprise one or more additional therapeutic agents including but not limited to DHA, EPA and omega-3 and their derivatives.

In certain embodiments, the endogenous compounds are isolated and/or purified or substantially purified by one or more purification methods described herein or known by those skilled in the art. Generally, the purities are at least 90%, in particular 95% and often greater than 99%. In certain embodiments, the naturally occurring compound is excluded from the general description of the broader genus.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. The term “pharmaceutically acceptable carriers” is intended to include all solvents, diluents, or other liquid vehicle, dispersion or suspension aids, surface active agents, isotonic agents, thickening or emulsifying agents, preservatives, solid binders, lubricants and the like, as suited to the particular dosage form desired. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention.

These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs are salts or prodrugs that are, within the scope of sound medical judgment, suitable for use in contact with the tissues of patients without undue toxicity, irritation, allergic response, and the like, commensurate with a reasonable benefit/risk ratio, and effective for their intended use. These compounds include the zwitterionic forms, where possible, of r compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “prodrug” refers to compounds that are rapidly transformed in vivo to yield the compounds of the invention, for example resolvins, protectins and their analogues and precursors, for example, by hydrolysis in blood. A thorough discussion is provided in T. Higachi and V. Stella, “Pro-drugs as Novel Delivery Systems,” Vol. 14 of the A.C.S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or Page 12 of 33 otherwise converted to the biologically, pharmaceutically or therapeutically active form of the compound. The prodrug may be designed to alter the metabolic stability or the transport characteristics of a compound, to mask side effects or toxicity, to improve the flavor of a compound or to alter other characteristics or properties of a compound. By virtue of knowledge of pharmacodynamic processes and drug metabolism in vivo, once a pharmaceutically active compound is identified, those of skill in the pharmaceutical art generally can design prodrugs of the compound (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. In one aspect, a solution of resolvin and/or protectin or precursor or analog thereof can be administered as eye drops for ocular neovascularization or ear drops to treat otitis.

Liquid dosage forms for oral administration of the compounds of the invention include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some instances, pharmaceutical compositions comprising the resolvins and protectins of the invention for the administration of angiogenesis may be in a formulation suitable for rectal or vaginal administration, for example as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compounds (resolvins and/or protectins and/or precursors or analogues thereof) of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

In particular embodiments, ophthalmic formulations for example, but not limited to eye ointments, eye drops, powders, solutions and injectable forms for ocular injections and the like are also encompassed within the scope of this invention. Such solutions are useful for the treatment of ocular neovascularization, for example but not limited to retinopathy of prematurity (ROP), diabetic retinopathy and age-related macular degeneration.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

More specifically, the invention features an article of manufacture that contains packaging material and at least one EPA or DHA analog contained within the packaging material. The packaging material contains a label or package insert indicating that the formulation can be administered to the subject with neovascularization in an amount, at a frequency, and for a duration effective treat or prevent symptoms associated with such disease states or conditions discussed throughout this specification. In some embodiments, the neovascularization is ocular neovascularization, for example diabetic retinopathy or age-related macular degeneration or retinopathy of prematurity.

Remington's Pharmaceutical sciences Ed. Germany, Merk Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; malt; gelatin; talc; excipients such as cocoa butter and: suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; water; isotonic saline; Ringer's solution, ethyl alcohol, and phosphate buffer solutions, as well as other non-toxic compatible lubricants such as sodium lauryl sulfate and magnesium sulfate, as well as coloring agents, releasing agents, coating agents, sweetening, flavoring and perfuming agents, preservatives and antioxidants can also be present in the composition, according to the judgment of the formulator.

### EXAMPLES

The examples presented herein relate to use or resolvins and protectins for the treatment and prevention of angiogenesis. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods

Animals. These studies adhered to the ARVO Statement for the Use of Animals in Ophthalmic and Vision Research. Fat-1 transgenic mice contain a humanized fat-1 cDNA driven by the cytomegalovirus enhancer and a chicken β-actin promoter⁹. Fat-1 and control dams were fed a defined diet with elevated omega-6 polyunsaturated fatty acids. For diet studies C57B116 mothers at delivery were fed a defined rodent diet with 10% (w/w) safflower oil containing 2% omega-6 polyunsaturated fatty acids (AA) and no omega-3 polyunsaturated fatty acids or the same defined diet except for 2% omega-3 polyunsaturated fatty acids (DHA and EPA) and no omega-6 polyunsaturated fatty acids (AA). (Table 1) Diets were stable over time and with oxygen exposure (Table 2).

O₂-induced retinopathy (vessel degeneration, re-growth and pathological neovascularization). To induce vessel loss, postnatal day 7 (P7) mice with their nursing mother were exposed to 75% oxygen for times ranging from 24 hours to 5 days¹. To evaluate vaso-obliteration following 24 hours of oxygen exposure, P8 mice were anesthetized at with Avertin (Sigma) and perfused with 50 μl of 120 mg/ml FITC-dextran (2×10⁶ molecular weight, FD2000S-5G, Sigma) in saline through the left ventricle²¹. Eyes were enucleated and fixed in 4% paraformaldehyde for 2 h at 4° C. Retinas were isolated and whole-mounted with SlowFade Antifade reagent (S2828, Molecular Probes) onto polylysin-coated slides with the photoreceptor side up. Retinas were examined with a fluorescence microscope (Olympus, Tokyo), digitized images using a three-charge-coupled device color video camera (DX-950P, Sony), and processed with NORTHERN ECLIPSE software (Empix Imaging, Toronto). Retinal neovascularization was evaluated 5 days after oxygen exposure (P7-P12) at P17 when the neovascular response is greatest. P17 mice were given a lethal dose of Avertin (Sigma) and their eyes were enucleated and fixed in 4% paraformaldehyde for 2 h at 4° C. Retinas were isolated and stained overnight with fluoresceinated Griffonia Bandereiraea Simplicifolia Isolectin B4 (Alexa Fluor 488-121411 or Alexa Fluor 594-121413, Molecular Probes) in 1 mM CaCl₂ in PBS. Following 2 hours of washes, retinas were whole-mounted with glycerol-gelatin (Sigma) onto polylysin-coated slides with the photoreceptor side up and imaged with a confocal microscope.

Quantification of vaso-obliteration and retinal neovascularization. Images of each of 4 quadrants of whole-mounted retina were taken at 5× magnification and imported into Adobe Photoshop. Retinal segments were merged to produce an image of the entire retina. Vaso-obliteration and neovascular tuft formation were quantified by comparing the number of pixels in the affected areas with the total number of pixels in the retina. Percentages of vaso-obliteration and neovascularization from mouse retinas were compared with values for retinas from age-matched control mice with identical oxygen conditions^{17,22}. Evaluation was done blind to the identity of the sample.

Resolvins and Neuroprotectins. C57B1/6 nursing mothers were fed a diet rich in either omega-6 or omega-3 polyunsaturated fatty acids from birth. To induce proliferative retinopathy in the pups, mice were exposed to 75% oxygen from P7 to P12. Retinas collected at P17 from omega-6 or omega-3 polyunsaturated fatty acid fed dams and exposed to high oxygen or room air conditions were analyzed to determine lipid mediator profiles. Polyunsaturated fatty acid derived products were extracted, identified, and quantified using a deuterium-labeled internal standard and MS-MS based mediator informatics²³. Results were obtained from retinas of six mice, each from a separate litter. Treatments with synthetic RvD1 (7S,8R,17S-trihydroxy-docosa-4Z,9E,11E,13Z,15E,19Z-hexaenoic acid), RvE1 (5S,12R,18R-trihydroxy-6Z,8E,10E,14Z,16E-eicosapentaenoic acid) and Neuroprotectin-D1 (10R,17S dihydroxy-docosa-4Z,7Z,11E,13E,15Z,19z-hexaneoic acid) were performed in 4 litters of mice. RvD1, RvE1 and NPD1 were prepared by organic synthesis according to published procedures matching physical and biological criteria^{14,24,25}. The pups were injected i.p. daily from P6-P17 (before, during, and after exposure to 75% oxygen) with 10 ng of either RvD1 (n=14), RvE1 (n=10) or NPD1 (n=14) or Saline/EtOH (n=14) vehicle. Following retinal staining and whole-mounting, neovascularization and vaso-obliteration were quantified at P17. Vaso-obliteration was also assessed at P8 (after 24 hours of 75% oxygen exposure) in pups given 10 ng of RvD1 (n=7), RvE1 (n=9), NPD1 (n=7), or saline/EtOH vehicle injections (n=6) i.p. from P5-P8. Vaso-obliteration was quantified on retinal whole-mounts following FITC-dextran intracardiac perfusion and fixation as above.

Quantitative analysis of gene expression (quantitative real-time PCR). PCR primers targeting Fat-1 and TNF-α and an unchanging control gene, cyclophilin were designed by using Primer Express software (Applied BioSystems, Foster City, Calif.). Three methods were used to analyze primer and probe sequences for specificity of gene detection. First, only primer and probe sequences that specifically detect the sequence of choice, as determined by means of the NCBI Blast module, were used. Second, amplicons generated during the PCR reaction were analyzed using the first derivative primer melting curve software supplied by Applied BioSystems. This analysis determined the presence of amplicons on the basis of their specific melting point temperatures. Third, amplicons generated during the PCR reaction were gel purified and sequenced (Children's Hospital Core Sequencing Facility, Boston, Mass.) to confirm the selection of the desired sequence. Quantitative analysis of gene expression was generated using an ABI Prism 7700 Sequence Detection System (TaqMan) and the SYBR Green master mix kit. Retinas were isolated from 6 mice per group and retinal RNA was isolated and converted to cDNA. (n=6 retinas per time point)

Lipid Extraction and Fatty Acid Analysis. Retinal samples containing 1 retina each were stored in buffered saline (10 mM Tris, 60 mM KCl, 30 mM NaCl, 2 mM Cl₂, 50 μM DTPA, 1.5 mM DTT and 1.5 ml/L aprotinin; adjusted to pH 8.0) at −80° C. until just prior to analysis. The samples were thawed and lipid extracted as previously described by Bligh and Dyer²⁶. Briefly, methanol containing 40 μg/ml butylated hydroxytoluene as an antioxidant was added to the retinal samples and chloroform was added to adjust the solvent ratios to 2:2:1.8 methanol/chloroform/water. The internal standard was 22:3n3 methyl ester (1.5 ug/mg tissue). Samples were homogenized for 30 sec using an Omni TH hand-held homogenizer. The homogenizer probe tip was cleaned in a solution containing chloroform/methanol/water between samples. Samples were vortexed for 1 min and centrifuged at 4° C. for 7 min at 3500 rpm (approx 2000×g) using a Sorvall RT7+ table-top centrifuge. The lower layer was collected. This procedure was repeated two times and the extracts pooled. The chloroform layer was then evaporated and then the samples were redissolved in chloroform. Half of the total lipid extract was taken for transmethylation according to the method of Morrison and Smith²⁷ as modified by Salem et al²⁸.

Methyl esters were quantified on a model 6890 series gas chromatograph (Agilent Technologies, Palo Alto, Calif.) using a FAST-GC method as described by Masood et al²⁹ using a 1 μl injection at a 25:1 split ratio. Tissue fatty acid methyl ester peak identification was performed by comparison to the peak retention times of a 28 component methyl ester standard (462, Nu-Chek Prep, Elysian, Minn.).

TNF-α receptor treatment. Intraperitoneal injections of a soluble TNF-α receptor (etanercept) (500 μg/mouse) were given at P7, P12, P14 and P16 to mice raised on and omega-6 rich diet as previously described³⁰. Retinas were isolated and stained with lectin-rhodamine at P17 to evaluate vaso-obliteration and retinal neovascularization. (n=6 mice per time point).

Intraocular injections. Mice with ischemic retinopathy were given an intravitreous injection of either etanercept (right eye) or a balanced salt solution (Alcon, left eye) on P12 after five days of 75% oxygen treatment. Each mouse received 0.5 microliters containing 12.5 μg of etanercept or saline (fellow eye). Injections were performed by inserting an Exmire microsyringe (MS-NE05, ITO Corp. Fuji, Japan) into the vitreous 1 mm posterior to the corneal limbus. Mice were anesthetized and their pupils were dilated with 1% tropicamide. Insertion and infusion were directly viewed through an operating microscope, taking care not to injure the lens or the retina. Retinal flatmounts of mice were analyzed 5 days post-injection at P17.

Western Blotting. Mice on an omega-3 or omega-6 polyunsaturated fatty acid diet were sacrificed and retinas were collected at P14. Retinas were homogenized and sonicated in 0.05 mM KPi buffer containing an array of phosphatase and protease inhibitors. Samples were normalized using a BSA assay (Pierce) and 50 μg of retinal lysate was loaded on a SDS-PAGE gel and then electroblotted onto PVDF membrane. The primary antibodies was rat anti-mouse TNF-α (Abeam), followed by an incubation with horseradish peroxidase-conjugated goat anti-rat IgG (Amersham) as the secondary antibodies. Antibodies were used according to manufactures recommendations. The primary antibody was applied overnight in 5% BSA at 4° C. Four mice per diet were used. Densitometry was analyzed using Image J.

Statistical Analysis. Results are presented as mean±SEM. for animal studies and mean±SD unless otherwise noted. ANOVA with α=0.05 was used for processing the data. A two-sample t test was used as a posttest unless otherwise indicated.

### Example 1

### Elevated Levels of Omega-3 Polyunsaturated Fatty Acids Result in Decreased Vaso-Obliteration and Retinopathy in Mice

Emerging knowledge of the properties of lipid mediators^{2,5}, as well as retrospective epidemiologic data describing polyunsaturated fatty acid-neovascular age-related macular degeneration relationships, suggests that EPA, DHA, and AA might act in vivo to regulate retinal vaso-obliteration and neovascularization⁵. To further investigate this possibility, the ability of moderate dietary intake of omega-3 polyunsaturated fatty acids or omega-6 polyunsaturated fatty acids to alter retinal angiogenesis was investigated. Mice on a defined isocaloric diet enriched with 2% of total fatty acids from either omega-3 polyunsaturated fatty acids (DHA and EPA) or omega-6 polyunsaturated fatty acid (AA), with their pups nursed with milk reflecting this diet were subjected to the model of oxygen induced retinopathy¹. In addition, the Fat-1 mouse⁹ which converts omega-6 polyunsaturated fatty acids to omega-3 polyunsaturated fatty acids to achieve an elevated omega-3 polyunsaturated fatty acid tissue status genetically was used in the same disease model. Tables 1-3 below indicate the Total Composition of Experimental Diets used in the experiments.

TABLE 1
Manufacture Diet Analysis Summary of Fatty Acid Compositions of Experimental
Diets.

| | | | Fatty Acid Profile (% TFA) | | | | | | | |
|---|---|---|---|---|---|---|---|---|---|---|
| Diet | # | General Description | OA | DHA | EPA | ALA | AA | LA | SA | PA |
| D03061805 | 2 | High oleate + ω-3 LCPUFA | 70 | 1 | 1 | ~0 | 0 | 11 | 2 | 6 |
| D04061502 | 4 | High oleate + ω-6 LCPUFA | ~70 | ~0 | ~0 | ~0 | ~2 | ~10 | ~2 | ~5 |
| NIH | 6 | NIH Diet (high in ω-6 precursors) | 24 | 1 | 0.1 | 2 | 0.1 | 55 | 3 | 8 |

Note:
AA = arachidonic acid (20:4ω-6);
ALA = a-linolenic acid (18:3ω-3).
Precursor to EPA DHA = docosahexaenoic acid (22:6ω-3);
EPA = eicosapentaenoic acid (20:5ω-3).
Precursor to DHA LA = linoleic acid (18:2ω-6).
Precursor to AA. Main ω-6 in diet;
OA = oleic acid (18:1ω-9). The high oleic acid diet was developed with the intention reducing AA precursors.
SA = stearic acid (18:0);
PA = palmitic acid (16:0);
% TFA = percent of total fatty acids

TABLE 2
AIN-93G Rodent Diet and Modified AIN-93G With Different Oils

| | Product # | | | |
|---|---|---|---|---|
| | ω-3 LCPUFA Feed | | ω-6 LCPUFA Feed | |
| | gm % | kcal % | gm % | kcal % |
| Protein | 20.3 | 19.6 | 20.3 | 19.6 |
| Carbohydrate | 61.0 | 58.8 | 61.0 | 58.8 |
| Fat | 10.0 | 21.7 | 10.0 | 21.7 |
| Total | | 100.0 | | 100.0 |
| kcal/gm | 4.15 | | 4.15 | |
| Ingredient | gm | kcal | gm | kcal |
| Casein, 80 Mesh | 0 | 0 | 0 | 0 |
| Casein, Alcohol | 200 | 800 | 200 | 800 |
| Extracted | | | | |
| L-Cystine | 3 | 12 | 3 | 12 |
| Corn Starch | 150 | 600 | 150 | 600 |
| Maltodextrin 10 | 150 | 600 | 150 | 600 |
| Sucrose | 100 | 400 | 100 | 400 |
| Dextrose | 200 | 800 | 200 | 800 |
| Cellulose, BW200 | 50 | 0 | 50 | 0 |
| Soybean Oil | 0 | 0 | 0 | 0 |
| Safflower Oil, High | 93 | 837 | 93 | 837 |
| Oleic | | | | |
| ROPUFA 30 | 5.7 | 51.3 | 0 | 0 |
| DHASCO (40% DHA) | 1.3 | 11.7 | 0 | 0 |
| ARASCO (40% AA) | 0 | 0 | 7 | 63 |
| t-BHQ | 0 | 0 | 0.014 | 0 |
| Mineral Mix S10022G | 35 | 0 | 35 | 0 |
| Vitamin Mix V10037 | 10 | 40 | 10 | 40 |
| Choline Bitartrate | 2.5 | 0 | 2.5 | 0 |
| Total | 1000.50 | 4152 | 1000.51 | 4152 |

TABLE 3
Fat-1 Feed: AIN-76A Rodent Diet and Same With 5% or 10%
Safflower Oil

Product #
Fat-1 Feed

| | gm % | kcal % |
|---|---|---|
| Protein | 21 | 21 |
| Carbohydrate | 59 | 58 |
| Fat | 10 | 22 |
| Total | | 100.0 |
| kcal/gm | 4.13 | |
| Ingredient | gm | kcal |
| Casein | 200 | 800 |
| DL-Methionine | 3 | 12 |
| Corn Starch | 150 | 600 |
| Sucrose | 401 | 1604 |
| Cellulose, BW200 | 50 | 0 |
| Corn Oil | 0 | 0 |
| Safflower Oil | 94 | 846 |
| Mineral Mix | 35 | 0 |
| S10001 | | |
| Vitamin Mix | 10 | 40 |
| V10001 | | |
| Choline Bitartrate | 2 | 0 |
| Total | 945 | 3902 |
| C2, Acetic | 0.00 | |
| C4, Butyric | 0.00 | |
| C6, Caproic | 0.00 | |
| C8, Caprylic | 0.00 | |
| C10, Capric | 0.00 | |
| C12, Lauric | 0.00 | |
| C14, Myristic | 0.00 | |
| C14:1, Myristoleic | 0.00 | |
| C16, Palmitic | 6.02 | |
| C16:1, Palmitoleic | 0.00 | |
| C18, Stearic | 2.16 | |
| C18:1, Oleic | 11.28 | |
| C18:2, Linoleic | 73.70 | |
| C18:3, Linolenic | 0.12 | |
| C18:4 | 0.00 | |
| C20, Arachidic | 0.00 | |
| C20:1, | 0.00 | |
| C20:4, Arachidonic | 0.00 | |
| C20:5, | 0.00 | |
| C22, Behenic | 0.00 | |
| C22:1, Erucic | 0.00 | |
| C22:4, | 0.00 | |
| Clupanodonic | | |
| C22:5 | 0.00 | |
| C22:6, | 0.00 | |
| C24, Lignoceric | 0.00 | |
| Total (gm) | 93.28 | |
| Saturated (g) | 8.2 | |
| Monounsaturated | 11.3 | |
| (g) | | |
| Polyunsaturated (g) | 73.8 | |
| Saturated (%) | 8.8 | |
| Monounsaturated | 12.1 | |
| (%) | | |
| Polyunsaturated (%) | 79.1 | |
| total n-6 (gm) | 73.7 | |
| total n-3 (gm) | 0.1 | |

Formulated from J. Nutr. 107:1340-1348, 1977 and J. Nutr. 110:1726, 1980 and on Sep. 29, 2003.

To ensure that retinal composition reflected differences in dietary intake of lipids, the lipid status on pups was first determined by Fast GC/FID analysis. More specifically, the retinal polyunsaturated fatty acid lipid status in pups at postnatal day seventeen (P17) nursed from birth by mothers on a diet enriched in either omega-3 or omega-6 polyunsaturated fatty acids, or in pups expressing the Fat-1 transgene on a high omega-6 polyunsaturated fatty acid diet, verses their wild type controls, was determined by Fast GC/FID analysis. Milk has been previously shown to reflect the lipid profile of the mother's diet^{8,10}. Both the EPA/DHA enriched diet or expression of the Fat-1 gene in the mother led to an increase in all of the principal omega-3 polyunsaturated fatty acids in the retinas of the milk fed pups including EPA, DPA, omega-3 and DHA (p≦0.005), and a substantial increase in the total omega-3 polyunsaturated fatty acids and a concomitant decrease in the omega-6/omega-3 LC polyunsaturated fatty acid ratio. As indicated below in Table 4, the Fat-1 expressing mice as well as the EPA/DHA supplemented group also had a corresponding decrease in retinal omega-6 polyunsaturated fatty acids including AA, DTA and DPA omega-6 (p≦0.005) and a decrease in the total retinal omega-6 polyunsaturated fatty acids relative to the AA supplemented group, as expected.

TABLE 4
Fatty acyl composition of retinas from P17 pups
Retinal Lipids at P17 (weight % of total fatty acids)

| Fatty Acid Family | ω-6 diet (n = 6) | ω-3 diet (n = 6) | Fat-1 WT (n = 6) | Fat-1 (n = 4) |
|---|---|---|---|---|
| Saturates | | | | |
| PA (16:0) | 22.53 (0.14) | 22.81 (0.24) | 21.40 (0.05) | 21.83 (0.88) |
| SA (18:0) | 20.30 (0.12) | 20.51 (0.26) | 19.01 (0.06) | 19.26 (0.14) |
| Total SFA | 44.85 (0.21) | 45.39 (0.43) | 41.84 (0.43) | 42.31 (1.14) |
| Monounsaturates | | | | |
| OA (18:1ω9) | 8.43 (0.03) | 8.79 (0.09)‡ | 6.61 (0.13) | 6.91 (0.62) |
| VA (18:1ω7) | 2.40 (0.03) | 2.21 (0.04)‡ | 2.01 (0.05) | 1.82 (0.13) |
| Total MUFA | 11.98 (0.10) | 12.15 (0.12) | 9.45 (0.21) | 9.61 (0.99) |
| ω-6 Polyunsaturates | | | | |
| LA (18:2ω6) | 0.74 (0.01) | 0.90 (0.04)‡ | 1.65 (0.04) | 1.76 (0.09) |
| AA (20:4ω6) | 8.87 (0.34) | 7.11 (0.35)‡ | 11.40 (0.21) | 8.41 (0.00)‡ |
| DTA (22:4ω6) | 1.25 (0.15) | 0.57 (0.09)‡ | 2.25 (0.03) | 0.85 (0.05)‡ |
| DPA (22:5ω6) | 4.29 (0.29) | 0.96 (0.08)‡ | 4.93 (0.12) | 0.29 (0.01)‡ |
| Total ω-6 PUFA | 15.82 (0.39) | 10.50 (0.30)‡ | 21.66 (0.28) | 12.69 (0.10)‡ |
| ω-3 Polyunsaturates | | | | |
| ALA (18:3ω3) | 0.03 (0.003) | 0.03 (0.01) | 0.01 (0.00) | 0.03 (0.01)‡ |
| EPA (20:5ω3) | 0.02 (0.0002) | 0.25 (0.02)‡ | 0.00 (0.00) | 0.52 (0.01)‡ |
| DPA (22:5ω3) | 0.17 (0.01) | 0.47 (0.03)‡ | 0.15 (0.01) | 0.76 (0.01)‡ |
| DHA (22:6ω3) | 12.65 (0.93) | 17.92 (1.07)‡ | 17.58 (0.22) | 26.58 (0.39)‡ |
| Total ω-3 PUFA | 12.87 (0.93) | 18.68 (1.07)‡ | 17.74 (0.22) | 27.93 (0.37)‡ |
| DHA/DPAω6 | 3.08 (0.40) | 19.63 (2.32)‡ | 3.57 (0.13) | 92.36 (5.14)‡ |
| ω-6/ω-3 ratio | 1.23 | 0.56 | 1.22 | 0.45 |

*Retinal lipids were compared in pups fed by dams on an ω-3 or ω-6 PUFA diet or in mice expressing the Fat-1 gene and their WT controls on a high ω-6 PUFA diet.
Statistical significance of these comparisons is represented in the ω-3 diet column: ‡ p ≦ 0.005 (standard deviation).
PA, palmitic acid;
SA, stearic acid;
SFA, saturated fatty acids;
OA, oleic acid;
VA, vaccenic acid;
MUFA, monounsaturated fatty acids;
LA, linoleic acid;
AA, arachidonic acid;
DTA, docosatetraenoic acid;
DPA, docosapentaenoic acid;
PUFA, polyunsaturated fatty acids;
ALA, alpha-linolenic acid;
EPA, eicosapentaenoic acid;
DHA, docosahexaenoic acid.

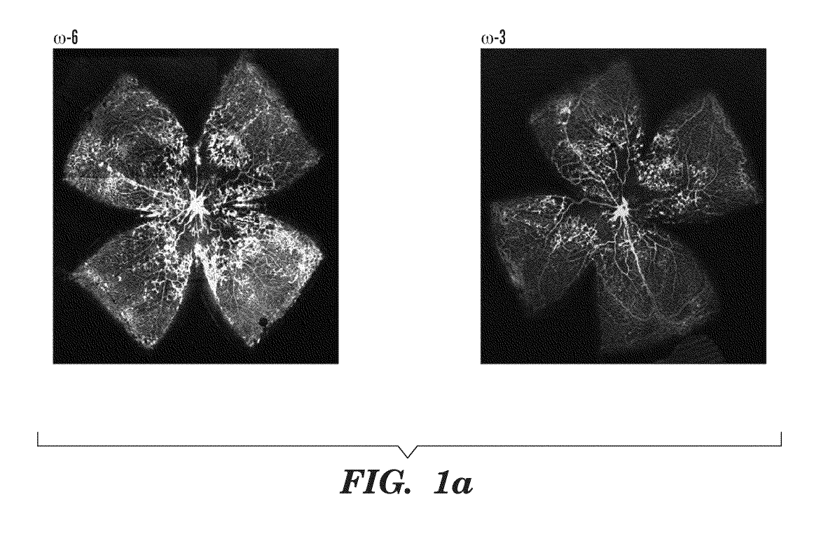

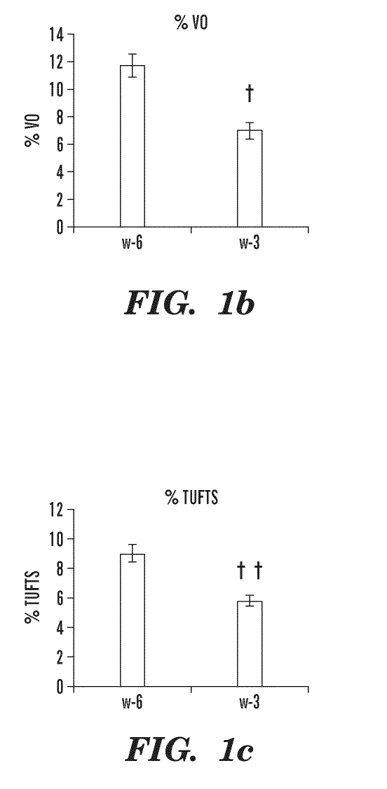

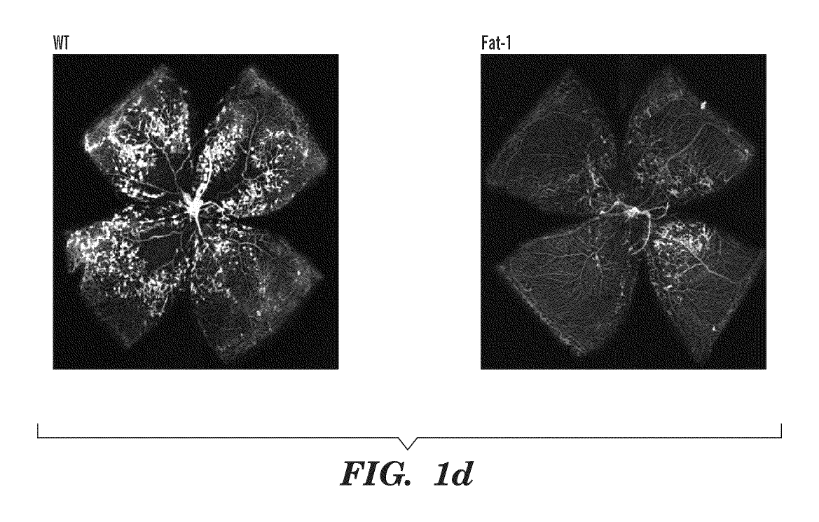

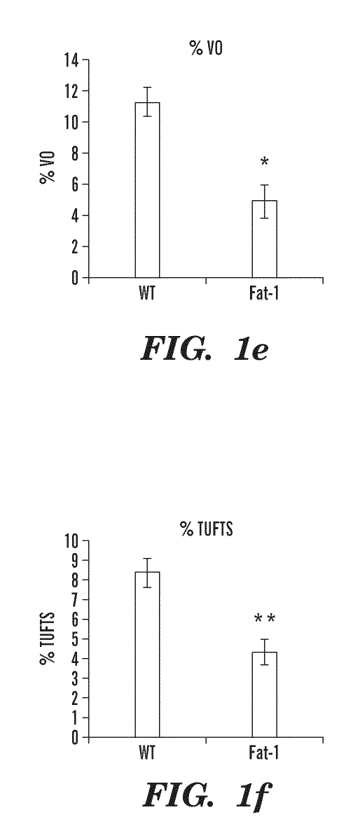

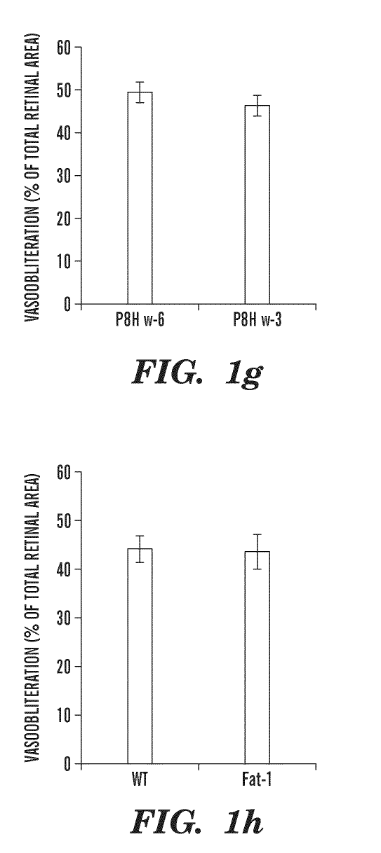

The effects against pathological angiogenesis of dietary modifications in omega-3 or omega-6 polyunsaturated fatty acids was first analyzed. At P17 mice subjected to conditions to generate oxygen induced retinopathy as per the model¹ were fed from dams on either the moderately enriched omega-6 polyunsaturated fatty acid diet, or on an omega-3 polyunsaturated fatty acid diet, and their retina were examined. The mice which received milk generated from a diet of moderately enriched omega-6 polyunsaturated fatty acid diet had a vaso-obliterated area of 11.7±3.2% (mean±S.E.M.) of total retinal area whereas the area of vaso-obliteration in mice on an omega-3 polyunsaturated fatty acid diet was 6.9±3.2% (†p≦0.0001, FIG. 1a, b). At P17 there was a significant protective effect from pathologic neovascularization in pups fed from dams on an omega-3 polyunsaturated fatty acid enriched diet (FIG. 1a, c). The mean neovascular growth in omega-3 polyunsaturated fatty acid fed mice was 5.7±2.0% of the total retinal area, compared to 9.0±2.3% (††p≦0.0001, FIG. 1c) for those on an omega-6 polyunsaturated fatty acid diet.

Mice expressing the fat-1 transgene which converts omega-6 to omega-3 polyunsaturated fatty acid were then used experimentally to validate effects on retinal neovascularization through manipulation of polyunsaturated fatty acids in diet. These mice have an elevated omega-3 polyunsaturated fatty acid and reduced omega-6 polyunsaturated fatty acid tissue level when fed an omega-3 polyunsaturated fatty acid deficient, omega-6 polyunsaturated fatty acid replete diet⁹. To evaluate the effect of polyunsaturated fatty acid changes on vessel survival and re-growth, Fat-1 mice and wild type controls were subjected to 75% oxygen from P7 to P12 to induce vessel loss¹. At P17 wild type mice lacking the fat-1 gene had extensive oxygen-induced vaso-obliteration (11.3±4.5% of total retinal area) as compared to fat-1 expressing mice (4.9±4.3%, *p<0.001; FIG. 1d, e). Hypoxia-induced retinal neovascularization is maximal in the model at P17¹. Following the induction of retinopathy, wild type mice at P17 had significantly more severe retinal neovascularization (8.3±3.3% of total retinal area) than did the fat-1 homozygotes (4.3±2.6% **p<0.001; FIG. 1d, f).

The results of the experiments described above indicated that elevation of omega-3 polyunsaturated fatty acid protected against retinal vaso-obliteration and retinal neovascularization at P17. Two possibilities regarding the protective mechanism existed; elevated omega-3 polyunsaturated fatty acid may have increased vessel re-growth or may have decreased oxygen-induced vessel loss. To assess the contribution of oxygen-induced vessel loss mice either on a omega-3 polyunsaturated fatty acid or omega-6 polyunsaturated fatty acid diet, or fat-1 mice and their wild-type controls subjected to oxygen induced retinopathy as described herein were assessed at P8 during hyperoxia exposure. The assessment revealed that elevated omega-3 polyunsaturated fatty acid by dietary intake or genetically in fat-1 mice did not protect against oxygen-induced vessel loss at P8 (FIG. 1g, h). These results indicate that the protective effect exerted by omega-3 polyunsaturated fatty acids against retinal neovascularization is mediated by enhanced vessel regrowth rather than through suppression of oxygen-induced vessel loss.

### Example 2

### ResolvinD1, ResolvinE1 and NeuroprotectinD1, Derived from Omega-3 Polyunsaturated Fatty Acids are Potent Protectors Against Retinopathy with Reduction In Vaso-Obliteration and Neovascularization

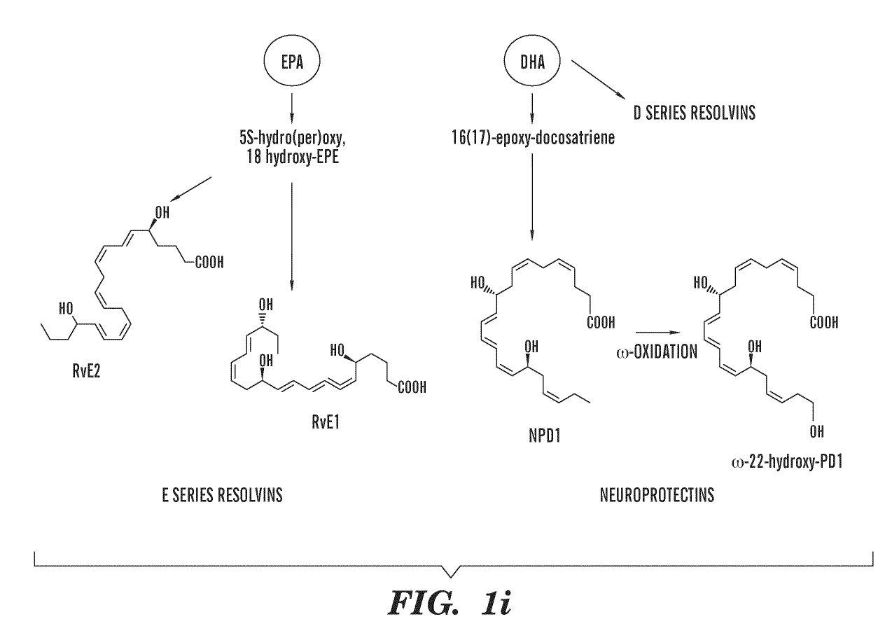

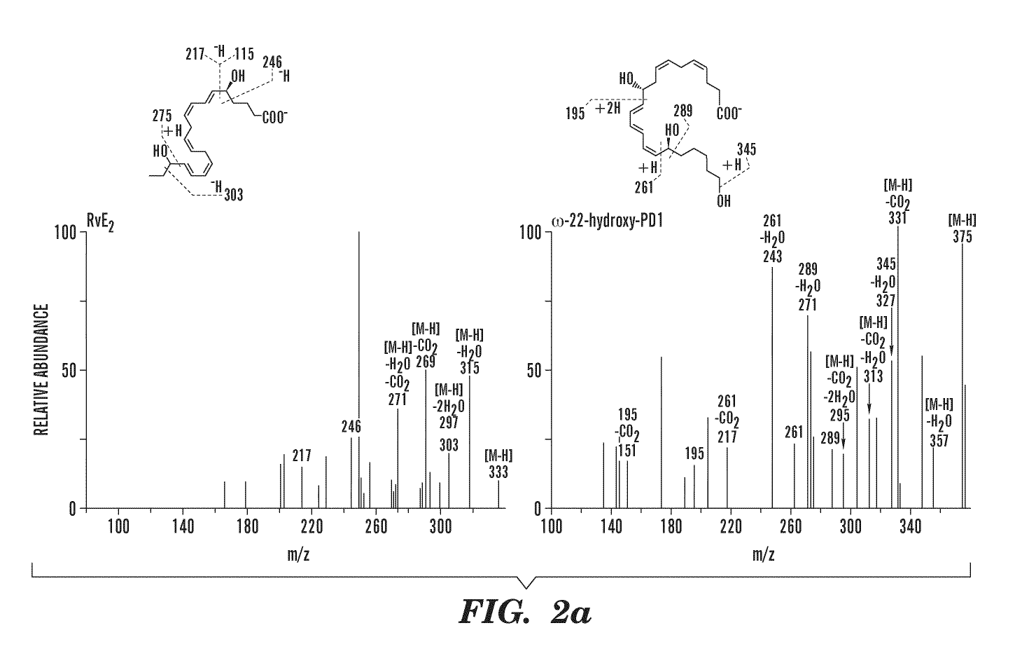

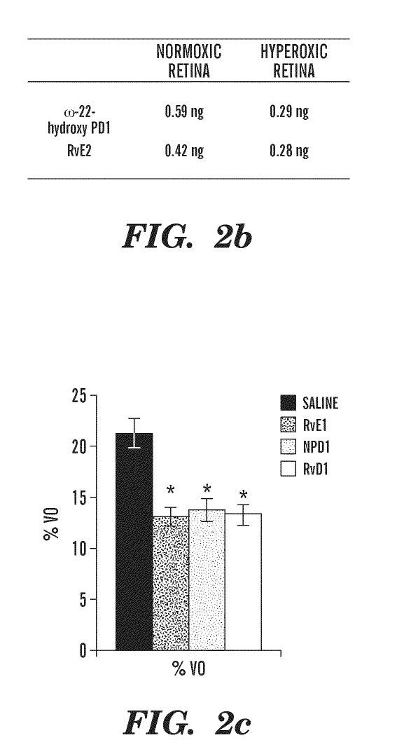

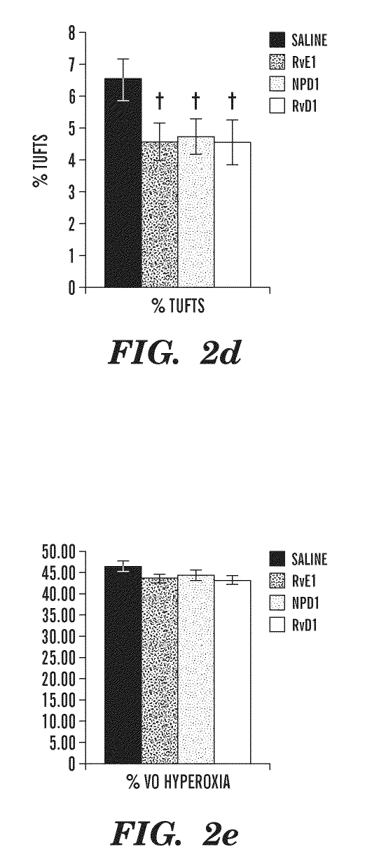

The resolvins (resolution phase interaction products) and neuroprotectins (including neuroprotectin D1, also known as protectin D1) are omega-3 polyunsaturated fatty acid bioactive products derived from EPA and DHA (FIG. 1 i) that were first identified in resolving inflammatory exudates in tissues enriched with DHA¹¹. The contribution to regulation of angiogenesis by resolvins and neuroprotectins has yet to be investigated¹¹. Retinas of pups fed from dams on diets rich in omega-3 or omega-6 polyunsaturated fatty acids were analyzed for the presence of resolvins and neuroprotectins. In the retinas of the mice pups fed from dams on an omega-6 polyunsaturated fatty acid diet, resolvin or neuroprotectin family members could not be detected. Conversely, in mice fed from dams given the omega-3 polyunsaturated fatty acid diet, omega-22-hydroxy-PD I and resolvinE2 (RvE2) were identified, each of which are biosynthetic pathway markers (FIG. 1i) formed in the biosynthesis of neuroprotectinD1 (NPD1) and resolvinE1 (RvE1) respectively (FIG. 2a, b)^{12,13}. To determine if these bioactive products mediated protective activities of omega-3 polyunsaturated fatty acids against retinopathy, the role of resolvin family members, resolvinD1 (RvD1) and RvE1 as well as the neuroprotectin NPD1, in vessel loss and neovascularization was assessed in the oxygen-induced retinopathy model. A very low dose of NPD1, RvD1, RvE1 (10 ng/day, comparable to levels found in the omega-3 polyunsaturated fatty acid-treated retinas in vivo (FIG. 2b)) or saline was administered intraperitoneally (i.p.) from P6-P17 in mice with oxygen-induced retinopathy. RvD1, RvE1 and NPD1 conferred significant protection from vaso-obliteration, compared to saline-injected controls (*p≦0.0001, FIG. 2c). In addition, less neovascularization at P17 in RvD1, RvE1 and NPD1 treated mice was observed compared to saline controls (†p≦0.03, FIG. 2d). To determine if the decrease in vaso-obliteration of RvD1, RvE1 and NPD1 treated mice was caused by enhanced vessel regrowth or prevention of vessel loss, mice were treated earlier during oxygen-induced vaso-obliteration from P5-P8. No apparent differences in vessel loss was observed between RvD1, RvE1 or NPD1 treated mice and the saline-injected control mice (FIG. 2e), indicating that these compounds confer their protective actions against retinopathy via enhanced vessel re-growth, and not via the suppression of vessel loss. These central findings are concordant with those from the dietary polyunsaturated fatty acid results presented above. Together they suggest that the effect of omega-3 polyunsaturated fatty acids on retinal neovascularization is consistent with actions, at least in part, with the biosynthesis of their potent bioactive mediators NPD1 and RvE1.

### Example 3

### Diets Rich in Omega-6 Polyunsaturated Fatty Acid Induce Increased Retinal TNF-α Expression And Retinopathy which is Reversed by Blocking TNF-α

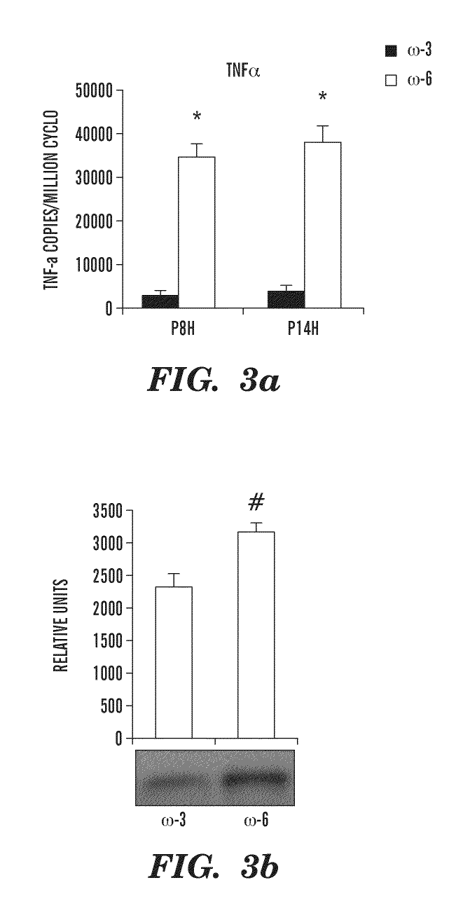

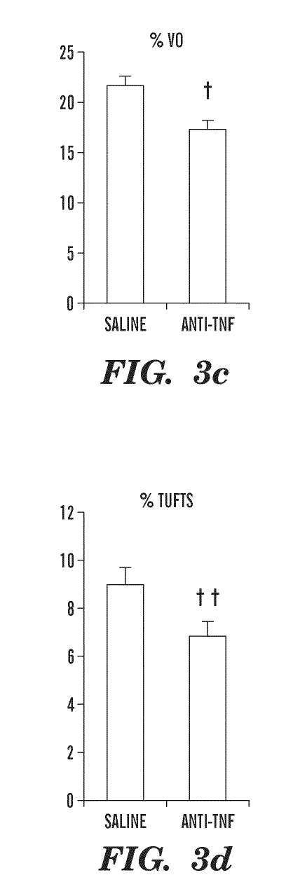

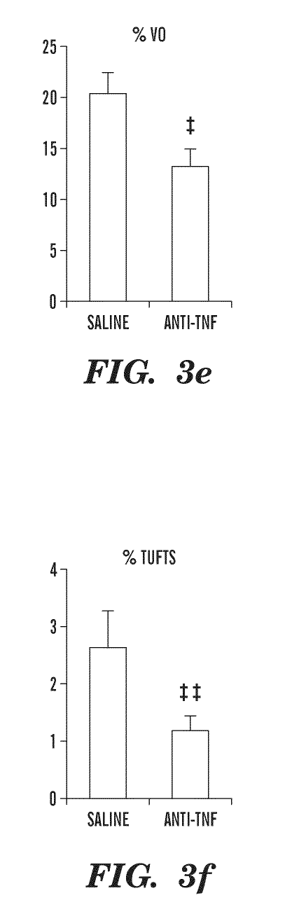

NPD1, RvD1 and RvE1 each significantly reduce TNF-α mRNA expression levels in inflammatory models^{14,15,16}. In addition, mice lacking TNF-α are protected from oxygen-induced retinopathy¹⁷. Given the above findings, the role of dietary intake of either omega-3 or omega-6 polyunsaturated fatty acids on retinal expression of TNF-α was explored by analysis of levels of TNF-α mRNA in pups fed from dams fed on the omega-3 diet and on the omega-6 diet following oxygen-induced retinopathy. The omega-3 polyunsaturated fatty acid diet potently suppresses TNF-α mRNA expression by ±90% at both P8 (hyperoxia) and P14 (hypoxia) compared to an omega-6 polyunsaturated fatty acid diet (*p≦0.0001, FIG. 3a). In addition, retinal levels of TNF-α protein were significantly reduced in pups fed by dams on an omega-3 polyunsaturated fatty acid diet relative to those fed by dams on an omega-6 polyunsaturated fatty acid diet (#p≦0.001, FIG. 3b). To further analyze the role of omega-6 polyunsaturated fatty acid on TNF-α during pathological neovascularization, TNF-α receptor fusion protein (etanercept) was injected i.p. to lower systemic TNF-α levels in omega-6 polyunsaturated fatty acid fed mice. Treatment with the TNF-α receptor fusion protein significantly protected pups on the omega-6 polyunsaturated fatty acid diet (with elevated levels of TNF-α) from vessel loss (†p≦0.001, FIG. 3c) as well as from pathologic neovascularization (††p≦0.05, FIG. 3d). This data suggests that the protective effect of omega-3 versus omega-6 polyunsaturated fatty acid diet was consistent with a relative increase in TNF-α in the pups of the omega-6 polyunsaturated fatty acid diet group. Intraocular injections of the TNF-α receptor fusion protein versus saline injection in the fellow eye also significantly reduced vaso-obliteration (†p≦0.003, FIG. 3e) and also suppressed retinal neovascularization (††p≦0.03, FIG. 30 in pups in the omega-6 polyunsaturated fatty acid diet group. It should be noted that any intraocular injections (control or treatment) greatly reduce neovascularization (unpublished findings).

The omega-3 (DHA, EPA) and omega-6 (AA) polyunsaturated fatty acids significantly influence vascular pathology. EPA and DHA and their potent bioactive products NPD1 and RvE1 and RvD1 at physiological levels promote vessel re-growth after vascular loss and injury as well as reduced pathologic neovascularization. Mice on an omega-6 polyunsaturated fatty acid diet have elevated levels of TNF-α which increases retinopathy. These effects on angiogenesis are important for a number of diseases such as diabetic retinopathy and retinopathy of prematurity as well as other pathologies where angiogenesis occurs. The omega-3 polyunsaturated fatty acid suppressive effect on retinopathy in the mouse eye is comparable in magnitude to anti-VEGF treatment¹⁸, and is likely to be additive to anti-VEGF therapy since VEGF is not significantly suppressed with the omega-3 polyunsaturated fatty acid diet.

These results suggest that enriching the sources of resolvins and protectins, and their precursors (including omega-3 polyunsaturated fatty acid) and their analogues are an effective therapeutic approach to help prevent proliferative retinopathy and angiogenesis. The present studies establish the first results indicating that resolvins RvE1 and RvD1 and neuroprotectin D1 (NPD1) are novel and potent regulators of angiogenesis.

### REFERENCES

The references cited herein and throughout the application are incorporated herein by reference.
1. Smith, L. E. et al. Oxygen-induced retinopathy in the mouse. Invest Opthalmol V is Sci 35, 101-11 (1994).
2. Kermorvant-Duchemin, E. et al. Trans-arachidonic acids generated during nitrative stress induce a thrombospondin-1-dependent microvascular degeneration. Nat Med 11, 1339-45 (2005).
3. Fierro, I. M., Kutok, J. L. & Serhan, C. N. Novel lipid mediator regulators of endothelial cell proliferation and migration: aspirin-triggered-15R-lipoxin A(4) and lipoxin A(4). J Pharmacol Exp Ther 300, 385-92 (2002).
4. Fliesler, S. J. & Anderson, R. E. Chemistry and metabolism of lipids in the vertebrate retina. Prog Lipid Res 22, 79-131 (1983).
5. SanGiovanni, J. P. & Chew, E. Y. The role of omega-3 long-chain polyunsaturated fatty acids in health and disease of the retina. Prog Retin Eye Res 24, 87-138 (2005).
6. Calder, P. C. Polyunsaturated fatty acids and inflammation. Prostaglandins Leukot Essent Fatty Acids (2006).
7. Salem, N., Jr., Litman, B., Kim, H. Y. & Gawrisch, K. Mechanisms of action of docosahexaenoic acid in the nervous system. Lipids 36, 945-59 (2001).
8. Serhan, C. N. & Savill, J. Resolution of inflammation: the beginning programs the end. Nat Immunol 6, 1191-7 (2005).
9. Kang, J. X., Wang, J., Wu, L. & Kang, Z. B. Transgenic mice: fat-1 mice convert n-6 to n-3 fatty acids. Nature 427, 504 (2004).
10. Moriguchi, T. et al. Effects of an n-3-deficient diet on brain, retina, and liver fatty acyl composition in artificially reared rats. J Lipid Res 45, 1437-45 (2004).
11. Serhan, C. N., Arita, M., Hong, S. & Gotlinger, K. Resolvins, docosatrienes, and neuroprotectins, novel omega-3-derived mediators, and their endogenous aspirin-triggered epimers. Lipids 39, 1125-32 (2004).
12. Hong, S., Gronert, K., Devchand, P. R., Moussignac, R. L. & Serhan, C. N. Novel docosatrienes and 17S-resolvins generated from docosahexaenoic acid in murine brain, human blood, and glial cells. Autacoids in anti-inflammation. J Biol Chem 278, 14677-87 (2003).
13. Tjonahen, E., Oh, S. F., Siegelman, J., Elangovan, S., Percarpio, K. B., Hong, S., Serhan, C. N. Resolvin E2: Identification and anti-inflammatory actions of a novel w-3 eicosapentaenoic acid-derived mediator. Cell. In Press. (2006).
14. Duffield, J. S. et al. Resolvin d series and protectin dl mitigate acute kidney injury. J Immunol 177, 5902-11 (2006).
15. Mukherjee, P. K., Marcheselli, V. L., Serhan, C. N. & Bazan, N. G. Neuroprotectin D1: a docosahexaenoic acid-derived docosatriene protects human retinal pigment epithelial cells from oxidative stress. Proc Natl Acad Sci USA 101, 8491-6 (2004).
16. Arita, M. et al. Resolvin E1, an endogenous lipid mediator derived from omega-3 eicosapentaenoic acid, protects against 2,4,6-trinitrobenzene sulfonic acid-induced colitis. Proc Natl Acad Sci USA 102, 7671-6 (2005).
17. Gardiner, T. A. et al. Inhibition of tumor necrosis factor-alpha improves physiological angiogenesis and reduces pathological neovascularization in ischemic retinopathy. Am J Pathol 166, 637-44 (2005).
18. Aiello, L. P. et al. Suppression of retinal neovascularization in vivo by inhibition of vascular endothelial growth factor (VEGF) using soluble VEGF-receptor chimeric proteins. Proc Natl Acad Sci USA 92, 10457-61 (1995).
19. Bannenberg, G. L. et al. Molecular circuits of resolution: formation and actions of resolvins and protectins. J Immunol 174, 4345-55 (2005).
20. Michels, S., Schmidt-Erfurth, U. & Rosenfeld, P. J. Promising new treatments for neovascular age-related macular degeneration. Expert Opin Investig Drugs 15, 779-93 (2006).
21. D'Amato, R., Wesolowski, E. & Smith, L. E. Microscopic visualization of the retina by angiography with high-molecular-weight fluorescein-labeled dextrans in the mouse. Microvasc Res 46, 135-42 (1993).
22. Banin, E. et al. T2-TrpRS inhibits preretinal neovascularization and enhances physiological vascular regrowth in OIR as assessed by a new method of quantification. Invest Opthalmol Vis Sci 47, 2125-34 (2006).
23. Lu, Y., Hong, S., Gotlinger, K. & Serhan, C. N. Lipid mediator informatics and proteomics in inflammation resolution. Scientific World Journal 6, 589-614 (2006).
24. Arita, M. et al. Stereochemical assignment, antiinflammatory properties, and receptor for the omega-3 lipid mediator resolvin E1. J Exp Med 201, 713-22 (2005).
25. Serhan, C. N. et al. Anti-inflammatory actions of neuroprotectin D1/protectin D1 and its natural stereoisomers: assignments of dihydroxy-containing docosatrienes. J Immunol 176, 1848-59 (2006).
26. Bligh, E.G. & Dyer, W. J. A rapid method of total lipid extraction and purification. Can J Biochem Physiol 37, 911-7 (1959).
27. Morrison, W. R. & Smith, L. M. Preparation of Fatty Acid Methyl Esters and Dimethylacetals from Lipids with Boron Fluoride—Methanol. J Lipid Res 5, 600-8 (1964).
28. Salem, N., Jr., Reyzer, M. & Karanian, J. Losses of arachidonic acid in rat liver after alcohol inhalation. Lipids 31 Suppl, S153-6 (1996).
29. Masood, A., Stark, K. D. & Salem, N., Jr. A simplified and efficient method for the analysis of fatty acid methyl esters suitable for large clinical studies. J Lipid Res 46, 2299-305 (2005).
30. Grounds, M. D. et al. Silencing TNFalpha activity by using Remicade or Enbrel blocks inflammation in whole muscle grafts: an in vivo bioassay to assess the efficacy of anti-cytokine drugs in mice. Cell Tissue Res 320, 509-15 (2005).

1. A method for the treatment of, or reducing the risk of, developing angiogenesis in a subject, the method comprising administering to the subject an effective amount of a pharmaceutical composition comprising a resolvin or agonists or analogues or precursors thereof.

2. A method for the treatment of, or reducing the risk of, developing angiogenesis in a subject, the method comprising administering to the subject an effective amount of a pharmaceutical composition comprising a protectin or agonists or analogues or precursors thereof.

3. The method of claim 1, wherein the resolvin is a di- or tri-hydroxy derivative of eicosapentaenoic acid (EPA) or deciashexaenoic acid (DHA).

4. The method of claim 3, wherein the hydroxy derivative of eicosapentaenoic acid (EPA) or deciashexaenoic acid (DHA) is an E-series resolvin or 18R resolvin of the E series.

5. The method of claim 3, wherein the E-series resolvin is selected from a group comprising; resolvin E1 (RvE1; (5S,12R,18R-trihydroxy-6Z,8E,10E,14Z,16E-eicosapentaenoic acid); 19-(p-fluorophenoxy)-RvE1; 18-oxo-RvE1; 5S,6R-epoxy,18R-hydroxy-EPE, Resolvin E2 (RvE2).

6. The method of claim 3, wherein the hydroxy derivative of eicosapentaenoic acid (EPA) or deciashexaenoic acid (DHA) is a D series resolvin or a 17R or 17S resolvin of the D-series.

7. The method of claim 3, wherein the D-series resolvin is selected from a group comprising; 17R-diH DHA; 16,17R-diH DHA; 17R-H(p) DHA; 7(8)-epoxy-17R-DHA; 4(5)-epoxy-17R-H DHA; Resolvin D1 (17S,8,17R-triDHA); resolvin D2 (17S,16,17-triDHA); Resolvin D3 (4S,11,17R-triDHA); Resolvin D4 (4S,5,17-triDHA).

8. The method of claim 2, wherein the protectin is a di- or tri-hydroxy derivative of deciashexaenoic acid (DHA).

9. The method of claim 8, wherein the hydroxy derivative of DHA is selected from a group comprising; neuroprotectin D1 (NPD1); protectin D1 (PD1); 10,17s-docosatriene or analogues and mimetics of NPD1; PD1 or 10,17s-docosatriene.

10. The method of claim 1, wherein the angiogenesis is associated with ocular neovascularization, tumor angiogenesis, arthritis, retinopathy, psoriasis, restenosis, capillary proliferation in atherosclerotic plaques.

11. The method of claim 10, wherein retinopathy is selected from a group comprising of: retinopathy of prematurity (ROP); diabetic retinopathy; age-related macular degeneration (AMD); retina vein occlusion; sickle cell retinopathy; Stargardt's disease; choroidal neovascularization, radiation retinopathy; symptoms associated with microangiopathy, ocular neovascularization, neovascular glaucoma.

12. The method of claim 2, wherein the angiogenesis is comprises ocular neovascularization, tumor angiogenesis, arthritis, retinopathy, psoriasis, restenosis, capillary proliferation in atherosclerotic plaques.

13. The method of claim 12, wherein retinopathy is limited to; retinopathy of prematurity (ROP); diabetic retinopathy; retina vein occlusion; sickle cell retinopathy; choroidal neovascularization, radiation retinopathy; symptoms associated with microangiopathy, ocular neovascularization, neovascular glaucoma.

14. The method of claim 10, wherein the retinopathy is retinopathy of prematurity.

15. The method of claim 10, wherein the subject is born preterm or the subject born before full gestation or weighing 10% less than the average for the subjects gestation age.

16.-31. (canceled)

32. The methods of claim 1, wherein the resolvins or agonists or analogues or precursors thereof are administered prophylatically or therapeutically.

33. (canceled)

34. The methods of claim 2, wherein the protectins or agonists or analogues or precursors thereof are administered prophylatically or therapeutically.

35.-39. (canceled)

40. The method of claim 12, wherein the retinopathy is retinopathy of prematurity.

41. The method of claim 12, wherein the subject is born preterm or the subject born preterm is a subject born before full gestation or weighing 10% less than the average for the subjects gestation age.
